(19) United States
(12) Patent Application Publication
(10) Pub. No.: US 2017/0007709 A1
(43) Pub. Date: Jan. 12, 2017

(54) GLYCOAMINO ACID AND USE THEREOF

(71) Applicant: AJINOMOTO CO., INC., Tokyo (JP)

(72) Inventors: Wataru KUROSAWA, Kawasaki-shi (JP); Risa UBAGAI, Kawasaki-shi (JP); Hiroyuki KATO, Kawasaki-shi (JP); Hiromi SUZUKI, Kawasaki-shi (JP)

(73) Assignee: AJINOMOTO CO., INC., Tokyo (JP)

(21) Appl. No.: 15/209,017

(22) Filed: Jul. 13, 2016

Related U.S. Application Data
(63) Continuation of application No. PCT/JP2015/051560, filed on Jan. 21, 2015.

(30) Foreign Application Priority Data
Jan. 21, 2014 (JP) 2014-009015

(51) Int. Cl. A61K 47/48; A61K 31/401; A61K 31/197; A61K 31/198; A61K 9/00; A61K 31/223

(52) U.S. Cl. CPC A61K 47/48092; A61K 9/0053; A61K 31/223; A61K 31/197; A61K 31/198; A61K 31/401

(57) ABSTRACT

An object of the present invention is to provide an amino acid precursor which shows improvement in the properties (particularly water-solubility, stability in water, bitter taste etc.) of amino acid, and can be converted to amino acid in vivo etc. The present invention relates to a compound for an amino acid precursor, which is a compound represented by the formula (I):

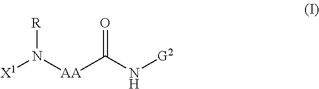

wherein each symbol is as described in the DESCRIPTION, or a salt thereof.

### CROSS REFERENCES TO RELATED APPLICATIONS

This application is a continuation of International Patent Application No. PCT/JP2015/051560, filed on Jan. 21, 2015, and claims priority to Japanese Patent Application No. 2014-009015, filed on Jan. 21, 2014, both of which are incorporated herein by reference in their entireties.

### BACKGROUND OF THE INVENTION

Field of the Invention

The present invention relates to a compound having improved property of amino acid and useful as an amino acid precursor which can be converted to amino acid in vivo and the like and use thereof.

Discussion of the Background

While amino acid is utilized for a broad range of applications, the application may be limited depending on the kind thereof due to the properties thereof. For example, since amino acids having low solubility in water (e.g., valine, leucine, isoleucine, tyrosine, cystine, phenylalanine, 3,4-dihydroxyphenylalanine etc.) cannot be easily dissolved in water at high concentrations, use thereof for aqueous compositions and liquid compositions is particularly subject to high restriction. When amino acids having low stability in water (e.g., cysteine, glutamine) are dissolved in water and used as liquid compositions and the like, the problems of decomposition, reaction of amino group with other components and the like, or the problems of coloration and odor tend to occur easily. In addition, amino acid with bitter tastes (e.g., valine, leucine, isoleucine) is under high restriction for oral application. As described above, since amino acid is restricted, due to its properties, particularly in the use as an aqueous composition and use for oral application, its use is sometimes difficult or formulation of a preparation requires some design.

On the other hand, a β-glucosyl amide derivative of a certain amino acid has been known. For example, non-patent document 1 discloses β-glucosyl amides of phenylalanine, aspartic acid and glutamic acid, which are synthesized via 4,6-O-benzylideneglucosylamine.

This document only discloses a synthesis method and does not at all disclose or suggest utility and usefulness of the above-mentioned β-glucosyl amides.

### DOCUMENT LIST

### Non-Patent Document

Non-patent document 1: J. Am. Chem. Soc., 83, (1961) pp. 1885-1888

### SUMMARY OF THE INVENTION

### Problems to be Solved by the Invention

An object of the present invention is to provide an amino acid precursor having improved property (particularly water-solubility, stability in water, bitter taste etc.) of amino acid, which can be converted to amino acid in vivo and the like.

### Means of Solving the Problems

The present inventors have conducted intensive studies in view of the above-mentioned problems and found that introduction of a group represented by the formula G²-NH—, wherein G² is a sugar residue wherein none of the hydroxyl groups are protected or modified, into a carboxy group of an amino acid to convert same to glycoamino acid or a salt thereof improves the properties (particularly water-solubility, stability in water, bitter taste etc.) that the amino acid itself has, and additionally the glycoamino acid or a salt thereof can be an amino acid precursor to be converted to amino acid in vivo etc., since a group represented by the above-mentioned formula G²-NH— detaches from amino acid in vivo etc., which resulted in the completion of the present invention. The present invention is as described below.

[1] A compound for an amino acid precursor which is a compound represented by the formula (I):

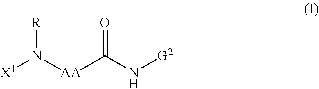

wherein
AA is an amino acid residue;
X¹ is a hydrogen atom, or a group represented by G¹-O—C(O)— (G¹ is a sugar residue wherein none of the hydroxyl groups are protected or modified);
G² is a sugar residue wherein none of the hydroxyl groups are protected or modified; and
R is a hydrogen atom or an alkyl group,
or a salt thereof (hereinafter to be also referred to as compound (I)).
[2] The compound for an amino acid precursor of the above-mentioned [1], wherein the sugar for the sugar residue wherein none of the hydroxyl groups are protected or modified for G¹ or G² is a monosaccharide.
[3] The compound for an amino acid precursor of the above-mentioned [1], wherein the sugar for the sugar residue wherein none of the hydroxyl groups are protected or modified for G² is glucose.
[4] The compound for an amino acid precursor of the above-mentioned [1], wherein the sugar for the sugar residue wherein none of the hydroxyl groups are protected or modified for G¹ is glucose, glucosamine or N-acetylglucosamine.
[5] The compound for an amino acid precursor of any of the above-mentioned [1]-[4], wherein R is a hydrogen atom.
[6] The compound for an amino acid precursor of the above-mentioned [1], wherein X¹ is a hydrogen atom and R is a hydrogen atom.
[7] The compound for an amino acid precursor of the above-mentioned [6], wherein the sugar for the sugar residue wherein none of the hydroxyl groups are protected or modified for G² is glucose.
[8] The compound for an amino acid precursor of any of the above-mentioned [1]-[7], wherein the amino acid of the amino acid residue for AA is α-amino acid.
[9] The compound for an amino acid precursor of any of the above-mentioned [1]-[7], wherein the amino acid of the amino acid residue for AA is valine, leucine, isoleucine, phenylalanine, tyrosine or 3,4-dihydroxyphenylalanine.
[10] The compound for an amino acid precursor of any of the above-mentioned [1]-[9], which is converted to amino acid in vivo.
[11] The compound for an amino acid precursor of any of the above-mentioned [1]-[10] for ingestion.
[12] A composition for ingestion comprising the compound for an amino acid precursor of any of the above-mentioned [1]-[11] and a carrier.
[13] The composition for ingestion of the above-mentioned [12], which is for oral application.
[14] A method of suppressing a bitter taste of amino acid, comprising introducing a group represented by the formula G²-NH—, wherein G² is a sugar residue wherein none of the hydroxyl groups are protected or modified, into a carboxy group of amino acid.
[15] The method of the above-mentioned [14], wherein the sugar for the sugar residue, wherein none of the hydroxyl groups are protected or modified, for G² is a monosaccharide.
[16] The method of the above-mentioned [14], wherein the sugar for the sugar residue, wherein none of the hydroxyl groups are protected or modified, for G² is glucose.
[17] The method of any of the above-mentioned [14]-[16], wherein the amino acid is α-amino acid.
[18] The method of any of the above-mentioned [14]-[16], wherein the amino acid is valine, leucine or isoleucine.
[19] The method of any of the above-mentioned [14]-[18], wherein the amino acid, wherein a group represented by the formula G²-NH— is introduced into a carboxy group, is converted to amino acid in vivo.
[20] A compound represented by

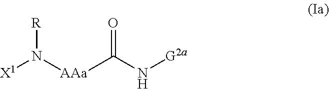

wherein
AAa is a residual group of amino acid selected from valine, leucine, isoleucine, tyrosine and 3,4-dihydroxyphenylalanine; X¹ is a hydrogen atom, or a group represented by G¹-O—C(O)— (G¹ is a sugar residue wherein none of the hydroxyl groups are protected or modified);
G^{2a} is a monosaccharide residue wherein none of the hydroxyl groups are protected or modified; and
R is a hydrogen atom or an alkyl group
or a salt thereof (hereinafter to be also referred to as compound (Ia)).
[21] The compound of the above-mentioned [20] or a salt thereof, wherein the sugar for the monosaccharide residue, wherein none of the hydroxyl groups are protected or modified, for G^{2a} is glucose.
[22] The compound of the above-mentioned [20] or [21] or a salt thereof, wherein the sugar for the sugar residue, wherein none of the hydroxyl groups are protected or modified, for G¹ is monosaccharide.
[23] The compound of the above-mentioned [20] or [21] or a salt thereof, wherein the sugar for the sugar residue, wherein none of the hydroxyl groups are protected or modified, for G¹ is glucose, glucosamine or N-acetylglucosamine.
[24] The compound of any of the above-mentioned [20]-[23] or a salt thereof, wherein R is a hydrogen atom.
[25] The compound of the above-mentioned [20] or a salt thereof, wherein X′ is a hydrogen atom and R is a hydrogen atom.
[26] The compound of the above-mentioned [25] or a salt thereof, wherein the sugar for the monosaccharide residue, wherein none of the hydroxyl groups are protected or modified, for G^{2a} is glucose.
[27] The compound of any of the above-mentioned [20]-[26] or a salt thereof, which is converted to amino acid in vivo.

### Effect of the Invention

A compound (glycoamino acid) wherein a group represented by the formula G²-NH— wherein G² is as defined above is introduced into a carboxy group of amino acid, or a salt thereof, shows improvement in the properties (particularly water-solubility, stability in water, bitter taste etc.) that the amino acid itself has, and the glycoamino acid or a salt thereof is highly useful as an amino acid precursor since the above-mentioned group represented by the formula G²-NH— is detached from amino acid in vivo etc. Therefore, the compound for an amino acid precursor of the present invention is particularly suitable for ingestion, and also suitable for oral application as an aqueous composition. Using such compound for an amino acid precursor of the present invention having improved water-solubility even in amino acid having comparatively high water-solubility, the broad utility of amino acid in the preparation of an aqueous composition or liquid composition for oral ingestion, and the like is markedly improved.

### BRIEF DESCRIPTION OF THE DRAWINGS

FIG. 1 shows amino acid production amounts from Leu-Glc by pronase.

FIG. 2 shows amino acid production amounts from Phe-Glc in an artificial bowel fluid.

FIG. 3 shows changes in the blood Leu concentration in rat by Leu or Leu-Glc administration.

FIG. 4 shows changes in the blood Val concentration in rat by Val or Val-Glc administration.

FIG. 5 shows changes in the blood Ile concentration in rat by Ile or Ile-Glc administration.

### DETAILED DESCRIPTION OF THE PREFERRED EMBODIMENTS

Unless otherwise specified in the sentences, any technical terms and scientific terms used in the present specification, have the same meaning as those generally understood by those of ordinary skill in the art the present invention belongs to. Any methods and materials similar or equivalent to those described in the present specification can be used for practicing or testing the present invention, and preferable methods and materials are described in the following. All publications and patents referred to in the present specification are hereby incorporated by reference so as to describe and disclose constructed products and methodology described in, for example, publications usable in relation to the described invention.

The present invention is explained in detail in the following.

AA shows an amino acid residue.

AAa shows a residual group of an amino acid selected from valine, leucine, isoleucine, tyrosine and 3,4-dihydroxyphenylalanine.

In the present specification, the “amino acid residue” for AA means a divalent group obtained by removing one amino group and one carboxy group from amino acid. The amino acid in the amino acid residue is not particularly limited as long as it has an amino group and a carboxy group, and may be any of α-amino acid, β-amino acid, γ-amino acid and the like. In AA, a side chain thereof may form a ring together with R, that is, the ring shown below.

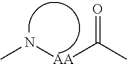

Examples of the α-amino acid include glycine, alanine, valine, leucine, isoleucine, serine, threonine, cysteine, methionine, glutamic acid, aspartic acid, lysine, arginine, histidine, glutamine, asparagine, phenylalanine, tyrosine, tryptophan, cystine, ornithine, thyroxin, proline, 3,4-dihydroxyphenylalanine and the like;

examples of the β-amino acid include β-alanine and the like; and
examples of the γ-amino acid include γ-aminobutyric acid and the like. When it has a functional group in the side chain, the functional group may be protected/modified as long as an adverse influence is not exerted on the properties (particularly water-solubility, stability in water, bitter taste etc.) of glycoamino acid.

Of these, α-amino acids such as valine, leucine, isoleucine, tyrosine, cystine, phenylalanine, 3,4-dihydroxyphenylalanine, cysteine, glutamine, glutamic acid, aspartic acid, lysine, proline and the like are preferable, and introduction of a group represented by the formula G²-NH— wherein G² is as defined above into a carboxy group is effective for the improvement of the above-mentioned properties in amino acids showing low solubility in water (e.g., valine, leucine, isoleucine, tyrosine, cystine, phenylalanine, 3,4-dihydroxyphenylalanine etc.), amino acids showing low stability in water (e.g., cysteine, glutamine etc.), and amino acids having bitter taste (e.g., valine, leucine, isoleucine etc.). Particularly, it is particularly effective for improving solubility in water and a bitter taste of valine, leucine and isoleucine.

The “residual group of an amino acid” of the “residual group of an amino acid selected from valine, leucine, isoleucine, tyrosine and 3,4-dihydroxyphenylalanine” for AAa means a divalent group obtained by removing one amino group and one carboxy group from the amino acid selected from valine, leucine, isoleucine, tyrosine and 3,4-dihydroxyphenylalanine.

The above-mentioned amino acid may be any of D form, L form and DL form.

X¹ is a hydrogen atom, or a group represented by G¹-O—C(O)— (G¹ is a sugar residue wherein none of the hydroxyl groups are protected or modified).

X¹ is preferably a hydrogen atom.

G² is a sugar residue wherein none of the hydroxyl groups are protected or modified.

G^{2a} is a monosaccharide residue wherein none of the hydroxyl groups are protected or modified.

In the present specification, “a sugar residue wherein none of the hydroxyl groups are protected or modified” for G¹ or G² means a moiety of a sugar wherein all hydroxyl groups are free, which excludes a hemiacetal hydroxyl group. The sugar residue may be modified/altered as long as all hydroxyl groups are free. Examples of the “sugar residue wherein none of the hydroxyl groups are protected or modified” include monosaccharides such as glucose, glucosamine, N-acetylglucosamine, mannose, galactose, fructose, ribose, lyxose, xylose, arabinose and the like; a moiety of saccharides such as polysaccharide composed of these monosaccharides and the like, which excludes a hemiacetal hydroxyl group.

In the present specification, “a monosaccharide residue wherein none of the hydroxyl groups are protected or modified” for G^{2a} means a moiety of a monosaccharide wherein all hydroxyl groups are free, which excludes a hemiacetal hydroxyl group. Examples of the “monosaccharide residue wherein none of the hydroxyl groups are protected or modified” include monosaccharides such as glucose, glucosamine, N-acetylglucosamine, mannose, galactose, fructose, ribose, lyxose, xylose, arabinose and the like, which excludes a hemiacetal hydroxyl group.

As G¹, a monosaccharide residue wherein none of the hydroxyl groups are protected or modified is preferable, a glucose residue, a glucosamine residue and an N-acetylglucosamine residue are more preferable, and a glucose residue is particularly preferable.

As G², a monosaccharide residue wherein none of the hydroxyl groups are protected or modified is preferable, a glucose residue, a glucosamine residue and an N-acetylglucosamine residue are more preferable, and a glucose residue is particularly preferable.

As G^{2a}, a glucose residue, a glucosamine residue and an N-acetylglucosamine residue are more preferable, and a glucose residue is particularly preferable.

The above-mentioned saccharide may be any of D form and L form, and D form present in large amounts in nature is preferable.

A partial structure represented by the formula G¹-O-which is formed from the above-mentioned saccharides may be an α-anomer structure, a β-anomer structure or a mixture thereof, and a β-anomer structure is preferable.

A partial structure represented by the formula G²-NH-which is formed from the above-mentioned saccharides may be an α-anomer structure, a β-anomer structure or a mixture thereof, and a β-anomer structure is preferable.

R is a hydrogen atom or an alkyl group.

The “alkyl group” for R is a C₁₋₁₀ alkyl group, more preferably a C₁₋₆ alkyl group. Specific preferable examples include methyl, ethyl, propyl, isopropyl, butyl, isobutyl, sec-butyl, tert-butyl, pentyl, hexyl and the like.

R is preferably a hydrogen atom.

Compound (I) is preferably a compound of the formula (I), wherein

AA is a valine residue, a leucine residue, an isoleucine residue, a phenylalanine residue, a tyrosine residue or a 3,4-dihydroxyphenylalanine residue;
X¹ is a hydrogen atom or a group represented by G¹-O—C(O)— (G¹ is a glucose residue, a glucosamine residue or an N-acetylglucosamine residue, wherein none of the hydroxyl groups are protected or modified);
G² is a glucose residue wherein none of the hydroxyl groups are protected or modified; and
R is a hydrogen atom,
or a salt thereof.

More preferably, it is a compound of the formula (I), wherein

AA is a valine residue, a leucine residue, an isoleucine residue, a phenylalanine residue, a tyrosine residue or a 3,4-dihydroxyphenylalanine residue;
X¹ is a hydrogen atom or a group represented by G¹-O—C(O)— (G¹ is a glucose residue wherein none of the hydroxyl groups are protected or modified);
G² is a glucose residue wherein none of the hydroxyl groups are protected or modified; and
R is a hydrogen atom,
or a salt thereof.

Further preferably, it is a compound of the formula (I), wherein

AA is a valine residue, a leucine residue, an isoleucine residue, a phenylalanine residue, a tyrosine residue or a 3,4-dihydroxyphenylalanine residue;
X¹ is a hydrogen atom;
G² is a glucose residue wherein none of the hydroxyl groups are protected or modified; and
R is a hydrogen atom,
or a salt thereof.

Of compounds (I), compound (Ia) is a novel compound.

Compound (Ia) is preferably a compound of the formula (Ia), wherein

AAa is a valine residue, a leucine residue, an isoleucine residue, a tyrosine residue or a 3,4-dihydroxyphenylalanine residue;
X¹ is a hydrogen atom or a group represented by G¹-O—C(O)— (G¹ is a glucose residue, a glucosamine residue or an N-acetylglucosamine residue, wherein none of the hydroxyl groups are protected or modified);
G^{2a} is a glucose residue wherein none of the hydroxyl groups are protected or modified; and
R is a hydrogen atom,
or a salt thereof.

More preferably, it is a compound of the formula (Ia), wherein

AAa is a valine residue, a leucine residue, an isoleucine residue, a tyrosine residue or a 3,4-dihydroxyphenylalanine residue;
X¹ is a hydrogen atom or a group represented by G¹-O—C(O)— (G¹ is a glucose residue wherein none of the hydroxyl groups are protected or modified);
G^{2a} is a glucose residue wherein none of the hydroxyl groups are protected or modified; and
R is a hydrogen atom,
or a salt thereof.

Further preferably, it is a compound of the formula (Ia), wherein

AAa is a valine residue, a leucine residue, an isoleucine residue, a tyrosine residue or a 3,4-dihydroxyphenylalanine residue;
X¹ is a hydrogen atom;
G^{2a} is a glucose residue wherein none of the hydroxyl groups are protected or modified; and
R is a hydrogen atom,
or a salt thereof.

While the production method of the compound for an amino acid precursor of the present invention is not particularly limited, for example, they can be synthesized by the following reactions.

Unless particularly indicated, the starting compound can be easily obtained as a commercially available product or can be produced by a method known per se or a method analogous thereto.

While the yield of the compound obtained by each of the following methods may vary depending on the reaction conditions to be used, the compound can be isolated and purified from the resultant products thereof by a conventional means (recrystallization, column chromatography etc.) and then precipitated by changing the solution temperature or solution composition and the like.

When an amino acid to be the starting compound in each reaction has a hydroxy group, an amino group, a carboxy group, a carbonyl group and the like on the side chain, a protecting group generally used in peptide chemistry and the like may be introduced into these groups, and the object compound can be obtained by removing the protecting group as necessary after the reaction.

Of compounds (I), compound (Ib) wherein X¹ is a hydrogen atom can be produced, for example, by the following steps.

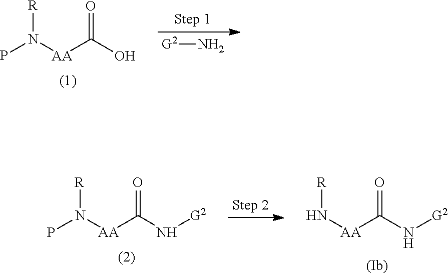

wherein P is an amino-protecting group, and other symbols are as defined above.

Examples of the amino-protecting group for P include a C₇₋₁₀ aralkyl-oxycarbonyl group (e.g., benzyloxycarbonyl), a C₁₋₆ alkoxy-carbonyl group (e.g., tert-butoxycarbonyl (Boc)), 9-fluorenylmethyloxycarbonyl (Fmoc) and the like.

### Step 1

In this step, a carboxy group of compound (1) or a salt thereof is reacted with G²-NH₂ to give compound (2).

This reaction is generally performed by reacting compound (1) or a salt thereof with chloroformic acid ester (e.g., methyl chloroformate, ethyl chloroformate, isobutyl chloroformate etc.) or pivaloyl chloride in a solvent that does not influence the reaction in the presence of a base to give the corresponding mixed anhydride, and reacting same with G²-NH₂.

As the base, triethylamine and the like can be mentioned.

The amount of the base to be used is generally 0.5-3 mol, preferably 1-2 mol, per 1 mol of compound (1) or a salt thereof.

While the solvent is not particularly limited as long as the reaction proceeds and, for example, ether (e.g., diethyl ether, diisopropyl ether, tert-butyl methyl ether, tetrahydrofuran, 1,4-dioxane, 1,2-dimethoxyethane etc.), halogenated hydrocarbon (e.g., chloroform, dichloromethane etc.), amides (e.g., dimethylformamide, dimethylacetamide etc.), N-methylpyrrolidone, acetonitrile, or a mixture thereof are used. Of these, tetrahydrofuran and a mixture of tetrahydrofuran and N-methylpyrrolidone are preferable.

The reaction temperature is generally −100-100° C., preferably −30-50° C., and the reaction time is generally for 0.5-30 hr, preferably for 1-5 hr.

Compound (1) or a salt thereof to be used may be a commercially available product or can also be produced by a conventionally-known method.

The thus-obtained compound (2) can be isolated and purified by a known separation and purification means, for example, concentration, concentration under reduced pressure, solvent extraction, crystallization, recrystallization, phase transfer, chromatography and the like. Compound (2) may be used without isolation for the next reaction.

### Step 2

In this step, the amino-protecting group P is removed from compound (2) to give compound (Ib) or a salt thereof.

When P is a benzyloxycarbonyl (Z) group, compound (2) is generally hydrogenated with a palladium catalyst in a solvent that does not influence the reaction.

As the palladium catalyst, palladium-carbon, palladium hydroxide and the like can be mentioned.

While the solvent is not particularly limited as long as the reaction proceeds and, for example, alcohol (e.g., methanol, ethanol etc.), ester (e.g., ethyl acetate) or a mixture thereof is used. Of these, methanol and ethyl acetate are preferable.

To accelerate the reaction, an adequate amount (e.g., 0.001%-30%) of an acid (e.g., hydrochloric acid, acetic acid, trifluoroacetic acid) can also be added.

When P is a tert-butoxycarbonyl (Boc) group, compound (2) is generally treated with an acid in a solvent that does not influence the reaction.

As an acid, hydrochloric acid, trifluoroacetic acid and the like can be mentioned.

While the solvent is not particularly limited as long as the reaction proceeds, for example, ether (e.g., diethyl ether, diisopropyl ether, tert-butyl methyl ether, tetrahydrofuran, 1,4-dioxane, 1,2-dimethoxyethane etc.), halogenated hydrocarbon (e.g., chloroform, dichloromethane etc.), amide (e.g., dimethylformamide, dimethylacetamide etc.), N-methylpyrrolidone, acetonitrile, or a mixture thereof is used. Of these, dioxane is preferable. An acid (e.g., hydrochloric acid, trifluoroacetic acid) can also be used as a solvent.

When P is a 9-fluorenylmethyloxycarbonyl (Fmoc) group, compound (2) is generally treated with a secondary amine in a solvent that does not influence the reaction.

As the secondary amine, piperidine, pyrrolidine, morpholine and the like can be mentioned.

While the solvent is not particularly limited as long as the reaction proceeds, for example, amide (e.g., dimethylformamide, dimethylacetamide etc.), halogenated hydrocarbon (e.g., chloroform, dichloromethane etc.), ether (e.g., diethyl ether, diisopropyl ether, tert-butyl methyl ether, tetrahydrofuran, 1,4-dioxane, 1,2-dimethoxyethane etc.), N-methylpyrrolidone, acetonitrile, or a mixture thereof is used. Of these, dimethylformamide is preferable.

The thus-obtained compound (Ib) or a salt thereof can be isolated and purified by a known separation and purification means, for example, concentration, concentration under reduced pressure, solvent extraction, crystallization, recrystallization, phase transfer, chromatography and the like.

Of compounds (I), compound (Ic) wherein X¹ is a group represented by G¹-O—C(O)— (G¹ is as defined above) and R is a hydrogen atom can be produced, for example, by the following steps.

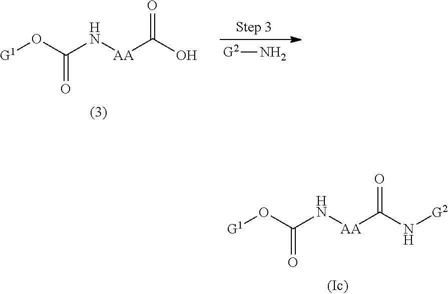

wherein each symbol is as defined above.

### Step 3

In this step, the carboxy group of compound (3) or a salt thereof is reacted with G²-NH₂ to give compound (Ic).

This step is performed by a method similar to that in step 1.

The thus-obtained compound (Ic) can be isolated and purified by a known separation and purification means, for example, concentration, concentration under reduced pressure, solvent extraction, crystallization, recrystallization, phase transfer, chromatography and the like.

Compound (3) which is a starting material for the above-mentioned step can be produced, for example, by the following method.

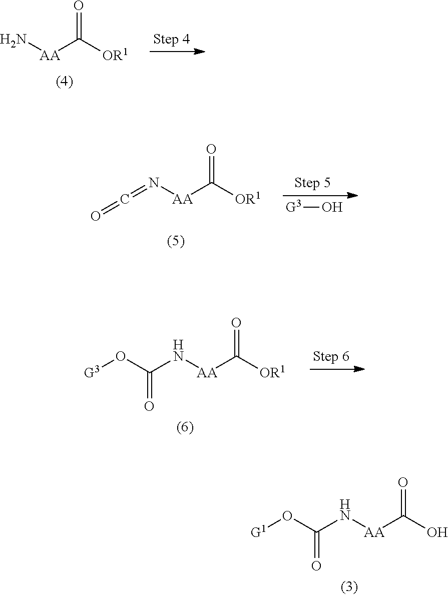

wherein R¹ is a carboxy-protecting group, G³ is a sugar residue wherein all hydroxyl groups are protected, and other symbols are as defined above.

Examples of the carboxy-protecting group for R¹ include C₁₋₆ alkyl group (e.g., methyl, ethyl, tert-butyl), C₇₋₁₄ aralkyl group (e.g., benzyl etc.), trisubstituted silyl group (e.g., trimethylsilyl, triethylsilyl, dimethylphenylsilyl, tert-butyldimethylsilyl, tert-butyldiethylsilyl etc.) and the like. Of these, methyl, ethyl and benzyl are preferable.

Examples of the sugar residue wherein all hydroxyl groups are protected for G³ include one wherein hydroxyl groups of “sugar residue wherein none of the hydroxyl groups are protected or modified” for G¹ are substituted by a protecting group such as C₇₋₁₄ aralkyl group (e.g., benzyl etc.), C₁₋₆ alkyl-carbonyl group optionally substituted by a halogen atom (e.g., acetyl, chloroacetyl), benzoyl group, C₇₋₁₄ aralkyl-carbonyl group (e.g., benzylcarbonyl etc.), 2-tetrahydropyranyl group, 2-tetrahydrofuranyl group, trisubstituted silyl group (e.g., trimethylsilyl, triethylsilyl, dimethylphenylsilyl, tert-butyldimethylsilyl, tert-butyldiethylsilyl etc.) and the like. Of these, acetyl and benzyl are preferable. It is preferable that all hydroxyl groups are protected by the same protecting group.

### Step 4

In this step, an amino group of compound (4) or a salt thereof is converted to an isocyanato group to give compound (5).

This reaction is generally performed by reacting compound (4) or a salt thereof with di-tert-butyl dicarbonate (Boc₂O) in the presence of a base in a solvent that does not influence the reaction.

The amount of di-tert-butyl dicarbonate to be used is generally 0.7-5 mol, preferably 1-2 mol, per 1 mol of compound (4) or a salt thereof.

Examples of the base include 4-(dimethylamino)pyridine and the like.

The amount of the base to be used is generally 0.5-3 mol, preferably 1-2 mol, per 1 mol of compound (4) or a salt thereof.

While the solvent is not particularly limited as long as the reaction proceeds, for example, hydrocarbon (e.g., benzene, toluene, xylene, hexane, heptane etc.), halogenated hydrocarbon (e.g., chloroform, dichloromethane etc.), ether (e.g., diethyl ether, diisopropyl ether, tert-butyl methyl ether, tetrahydrofuran, 1,4-dioxane, 1,2-dimethoxyethane etc.) or a mixture thereof is used. Of these, dichloromethane is preferable.

The reaction temperature is generally −100 to 100° C., preferably −30 to 50° C., and the reaction time is generally for 0.5-30 hr, preferably for 1-5 hr.

After completion of the reaction, compound (5) is subjected to the next step in the form of a reaction mixture without isolation.

When compound (4) is in the form of an acid addition salt, it is treated with a base to be converted to a free form, and subjected to this step or reacted in the presence of excess base.

### Step 5

In this step, compound (5) is reacted with G³-OH to give compound (6). G³-OH is a sugar wherein all hydroxyl groups other than hemiacetal hydroxyl group are protected.

This reaction is generally performed by reacting compound (5) with G³-OH in a solvent that does not influence the reaction.

The amount of G³-OH to be used is generally 0.7-5 mol, preferably 1-2 mol, per 1 mol of compound (5).

While the solvent is not particularly limited as long as the reaction proceeds, for example, hydrocarbon (e.g., benzene, toluene, xylene, hexane, heptane etc.), halogenated hydrocarbon (e.g., chloroform, dichloromethane etc.), ether (e.g., diethyl ether, diisopropyl ether, tert-butyl methyl ether, tetrahydrofuran, 1,4-dioxane, 1,2-dimethoxyethane etc.) or a mixture thereof is used. Of these, dichloromethane is preferable.

The reaction temperature is generally −100-100° C., preferably −30-50° C. and the reaction time is generally for 3-40 hr, preferably for 10-30 hr.

The thus-obtained compound (6) can be isolated and purified by a known separation and purification means, for example, concentration, concentration under reduced pressure, solvent extraction, crystallization, recrystallization, phase transfer, chromatography and the like. Compound (6) may be used for the next reaction without isolation.

### Step 6

In this step, the carboxy-protecting group R¹ of compound (6) and the hydroxyl-protecting group present in G³ are removed to give compound (3) or a salt thereof.

Removal of the carboxy-protecting group R¹ and removal of the hydroxyl-protecting group present in G³ may be performed simultaneously or in separate steps. In the latter case, the order thereof is not questioned but conveniently performed simultaneously. In this case, these protecting groups are selected to permit removal under the same conditions. For example, when the carboxy-protecting group R¹ is methyl or ethyl, and the hydroxyl-protecting group present in G³ is acetyl, they are removed by alkali hydrolysis.

Alkali hydrolysis is generally performed by treating compound (6) with alkali in a solvent that does not influence the reaction.

Examples of the alkali include lithium hydroxide, sodium hydroxide, potassium hydroxide, barium hydroxide and the like, and lithium hydroxide is preferable.

While the solvent is not particularly limited as long as the reaction proceeds, for example, water, alcohol (e.g., methanol, ethanol, isopropyl alcohol, tert-butyl alcohol etc.), ether (e.g., diethyl ether, diisopropyl ether, tert-butyl methyl ether, tetrahydrofuran, 1,4-dioxane, 1,2-dimethoxyethane etc.), halogenated hydrocarbon (e.g., dichloromethane etc.) or a mixture thereof is used. Of these, a mixture of water and alcohols (e.g., methanol, ethanol, isopropyl alcohol, tert-butyl alcohol etc.) is preferable.

The reaction temperature is generally −100-100° C., preferably −30-35° C. and the reaction time is generally for 5-10 hr, preferably for 0.5-2 hr.

The thus-obtained compound (3) or a salt thereof can be isolated and purified by a known separation and purification means, for example, concentration, concentration under reduced pressure, solvent extraction, crystallization, recrystallization, phase transfer, chromatography and the like.

Of compounds (I), a compound wherein X¹ is a group represented by G¹-O—C(O)— (G¹ is as defined above) and R is an alkyl group can be obtained by introducing an alkyl group into compound (6) by a known method, and removing the protecting group in the same manner as in step 6. Examples of the method for introducing an alkyl group include a method including reacting compound (6) introduced with a base-resistant protecting group with the corresponding alkyl halide under appropriate basic conditions. Alternatively, an alkyl group is introduced in advance into amino group of compound (4) by a known method, and compound (I) can be obtained by a method similar to steps 4, 5 and 6.

The thus-obtained compound (I) can be isolated and purified by a known separation and purification means, for example, concentration, concentration under reduced pressure, solvent extraction, crystallization, recrystallization, phase transfer, chromatography and the like.

Compound (I) may be used in the form of a metal salt or a salt with an organic base, where necessary. When compound (I) is in the form of a salt, such salt is preferably an edible salt. For example, metal salt, ammonium salt, salt with organic base, salt with inorganic acid, salt with organic acid, salt with basic or acidic amino acid and the like can be mentioned. Preferable examples of the metal salt include alkali metal salts such as potassium salt, sodium salt and the like; alkaline earth metal salts such as calcium salt, magnesium salt, barium salt and the like; aluminum salt and the like. Preferable examples of the salt with organic base include salts with triethylamine, trimethylamine, picoline, pyridine, 2,6-lutidine, ethanolamine, diethanolamine, triethanolamine, cyclohexylamine, dicyclohexylamine, N,N′-dibenzylethylenediamine and the like. Preferable examples of the salt with inorganic acids include salts with hydrochloric acid, hydrobromic acid, nitric acid, sulfuric acid, phosphoric acid and the like. Preferable examples of the salt with organic acid include salts with formic acid, acetic acid, trifluoroacetic acid, phthalic acid, fumaric acid, oxalic acid, tartaric acid, maleic acid, citric acid, malic acid, succinic acid, methanesulfonic acid, benzenesulfonic acid, p-toluenesulfonic acid and the like. Preferable examples of the salt with basic amino acid include salts with arginine, lysine, ornithine and the like, and preferable examples of the salt with acidic amino acid include salts with aspartic acid, glutamic acid and the like.

In compound (I), since a group represented by the formula G²-NH— wherein G is as defined above is introduced into a carboxy group of the amino acid, the properties (particularly water-solubility, stability in water, bitter taste etc.) that the amino acid itself has are improved. Therefore, improvement of water-solubility and stability in water expands application as an aqueous composition, and improvement of bitter taste renders the compound suitable for oral application.

In addition, since a group represented by the above-mentioned formula G²-NH— is detached from amino acid by bowel fluid and pronase, and a group represented by the above-mentioned formula G¹-O—C(O)— is detached from amino acid under acidic conditions of gastric juice and the like or by glucosidase (particularly β-glucosidase), compound (I) can be converted to amino acid in vivo, in soil and the like. Therefore, compound (I) is useful as an amino acid precursor. It is also useful as a sustained-release amino acid precursor which is continuously converted to amino acid.

Since compound (I) is particularly useful as an amino acid precursor that can be converted to amino acid in vivo and the like, it can be preferably used for ingestion. Also, compound (I) can be used for medicament and food as a composition for ingestion containing an amino acid precursor together with a carrier conventionally used in the fields of medicament and food.

Examples of the carrier used for the composition for ingestion of the present invention include

binders such as tragacanth, gum arabic, cornstarch, gelatin, polymer polyvinylpyrrolidone and the like;
excipients such as cellulose and a derivative thereof (e.g., microcrystalline cellulose, crystalline cellulose, hydroxypropyl cellulose etc.) and the like;
swelling agents such as cornstarch, pregelatinized starch, alginic acid, dextrin and the like;
lubricants such as magnesium stearate and the like;
flowability improving agents such as particle silicon dioxide, methyl cellulose and the like;
lubricants such as glycerin fatty acid ester, talc, polyethylene glycol 6000 and the like;
thickeners such as sodium carboxymethyl cellulose, carboxyvinyl polymer, xanthan gum, gelatin and the like;
sweetening agents such as sucrose, lactose, aspartame and the like;
flavors such as peppermint flavor, vanilla flavor, cherry flavor, orange flavor and the like;
emulsifiers such as monoglyceride, polyglycerin fatty acid ester, sucrose fatty acid ester, lecithin, polyoxyethylene hydrogenated castor oil, polyoxyethylene monostearic acid ester and the like;
pH adjusters such as citric acid, sodium citrate, acetic acid, sodium acetate, sodium hydroxide and the like;
thickeners such as sodium carboxymethyl cellulose, carboxyvinyl polymer, xanthan gum, gelatin and the like;
corrigents such as aspartame, licorice extract, saccharin and the like;
antioxidants such as vitamin C, vitamin A, vitamin E, various polyphenol, hydroxytyrosol, antioxidative amino acid, erythorbic acid, butylated hydroxyanisole, propyl gallate and the like;
preservatives such as sodium benzoate, sodium edetate, sorbic acid, sodium sorbate, methyl p-hydroxybenzoate, butyl p-hydroxybenzoate and the like;
colorants such as red iron oxide, yellow iron oxide, black iron oxide, carmine, Food Color Blue No. 1, Food Color Yellow No. 4, Food Color Red No. 2 and the like;
n-3 based fatty acids such as α-linolenic acid, eicosapentaenoic acid, docosahexaenoic acid and the like (fatty acid having a double bond between third and fourth carbons counted from the methyl group side of fatty acid);
fats and oils such as soybean oil, safflower oil, olive oil, corn oil, sunflower oil, Japanese basil oil, flaxseed oil, perilla oil, rape seed oil and the like;
coating agents such as shellac, sugar, hydroxypropylmethylcellulose phthalate, polyacetin and the like;
preservatives such as methylparaben, propylparaben and the like;
vitamins such as vitamin A, vitamin B group, vitamin C, vitamin D, vitamin E, nicotinic acid amide, folic acid, pantothenic acid, biotin, choline and the like;
various amino acids and the like.

When the composition for ingestion of the present invention is provided as an oral medicament, the form thereof is not particularly limited and, for example, liquid, tablet, granule, powder, capsule (including soft capsule), elixir, syrup, microcapsule, drink, emulsion, suspension and the like can be mentioned; and when it is provided as a parenteral medicament, the form thereof is not particularly limited and, for example, injection, infusion, drip infusion and the like can be mentioned. When the composition for ingestion of the present invention is provided as food or drink, the form thereof is not particularly limited and, for example, powder product, granular product, capsule product, tablet product, liquid product (e.g., drinks etc.), jelly-like drink, jelly-like product (e.g., jelly etc.), gum-like product, sheet-like product, solid-like product (e.g., snack bar, cookie etc.) and the like can be mentioned.

The composition for ingestion of the present invention can have a form containing a single ingestion amount packed or filled therein. For such packing, packing materials and packing methods (e.g., portion packing, stick packing etc.) generally used for packing medicament or food can be used. For such filling, a fill method generally used for medicament or food can be used. In the present specification, the “single ingestion amount” is, for example, the amount of the composition to be administered at one time when the composition for ingestion of the present invention is a medicament, and the amount of the composition to be ingested in one meal when the composition for ingestion of the present invention is food or drink. The single ingestion amount can be appropriately controlled according to the age, body weight, sex and the like of the subject who ingests.

In the composition for ingestion of the present invention, compound (I) may be contained singly or in any combination, and the amount thereof is not particularly limited and varies depending on the form. For example, it is preferably 1-70 wt %, more preferably 10-50%, particularly preferably 20-40%.

The composition for ingestion of the present invention can also be prepared according to the descriptions in JP-A-2010-59120, JP-A-2007-314497, JP-A-2005-289928, JP-A-2-128669, JP-B-3211824, JP-A-2002-187840, JP-A-2003-221329, WO 2004/019928, WO 2010/029951, JP-A-8-198748, JP-A-8-73351 and the like, and can also be applied to the form and use described therein.

### EXAMPLES

The present invention is explained in more detail in the following by referring to Examples, which are not to be construed as limiting the scope of the present invention in any way. The reagents, apparatuses and materials used in the present invention are commercially available unless otherwise specified.

In the Examples,

XXX-Glc means a glycoamino acid wherein the carboxy group at the α-position of amino acid (XXX) is amidated with a D-glucopyranosylamino group,

Glc-XXX-Glc means a glycoamino acid wherein the carboxy group at the α-position of amino acid (XXX) is amidated with a D-glucopyranosylamino group, and the amino group at the α-position is carbamated with a D-glucopyranosyloxycarbonyl group.

In the present specification, when amino acid and the like are indicated by abbreviations, each indication is based on the abbreviation of the IUPAC-IUB Commission on Biochemical Nomenclature or conventional abbreviations in the art.

For example, amino acid (XXX) is indicated as follows.
Leu: L-leucine
Phe: L-phenylalanine
Tyr: L-tyrosine
Gly: glycine
Ala: L-alanine
Val: L-valine
Ile: L-isoleucine
Ser: L-serine
Lys: L-lysine
Pro: L-proline
Thr: L-threonine
Met: L-methionine
Glu: L-glutamic acid
Cys: L-cysteine
Asp: L-aspartic acid
Gln: L-glutamine
Trp: L-tryptophan
His: L-histidine
Arg: L-arginine
DOPA: 3,4-dihydroxy-L-phenylalanine

In the following Examples, “room temperature” shows generally about 10° C. to about 35° C. The ratio shown for mixed solvents is a volume mixing ratio unless otherwise specified.

¹H-NMR (proton nuclear magnetic resonance spectrum) was measured by Fourier-transform NMR. When protons of hydroxy group, carboxy group, amino group and the like have very mild peaks, they are not described.

### Example 1

### Glc-Leu-Glc; N—(N-(α/β-D-glucopyranosyloxycarbonyl)-L-leucyl)-β-D-glucopyranosylamine

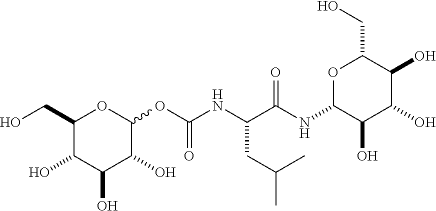

### (1) 4Ac-Glc-Leu-OMe; N-(2,3,4,6-tetra-O-acetyl-α/β-D-glucopyranosyloxycarbonyl)-L-leucine methyl ester

L-leucine methyl ester hydrochloride (Leu-OMe hydrochloride) (293 mg, 1.61 mmol) was suspended in tetrahydrofuran (3.5 ml), and the suspension was cooled in an ice bath. To this suspension was added triethylamine (4.3 ml, 30.8 mmol), and the mixture was warmed to room temperature and stirred for 30 min. The reaction solution was filtered, and concentrated to give L-leucine methyl ester (232 mg, 1.61 mmol).

Boc₂O (493 mg, 2.26 mmol) was dissolved in dichloromethane (10 ml), and the mixture was cooled in an ice bath. To this solution were added a solution of 4-(dimethylamino)pyridine (198 mg, 1.62 mmol) in dichloromethane (7 ml) and a solution of L-leucine methyl ester (232 mg, 1.61 mmol) in dichloromethane (7 ml), and the mixture was stirred at room temperature for 1 hr. The reaction solution was cooled again in an ice bath, a solution of 2,3,4,6-tetra-O-acetyl-D-glucose (787 mg, 2.26 mmol) in dichloromethane (10 ml) was added, and the mixture was stirred for 18 hr. The reaction solution was concentrated under reduced pressure, and the residue was purified by silica gel column chromatography (gradient; hexane:ethyl acetate=85:15→60:40) to give 4Ac-Glc-Leu-OMe (698 mg, 1.34 mmol, yield 83%) as a white powder.

¹H-NMR (400 MHz, CDCl₃) δ: 0.88-1.00 (m, 6H), 1.49-1.78 (m, 3H), 2.01 (s, 3H), 2.03 (s, 3H), 2.04 (s, 1.5H), 2.07 (s, 1.5H), 2.09 (s, 1.5H), 2.10 (s, 1.5H), 3.74 (s, 1.5H), 3.76 (s, 1.5H), 3.79-3.87 (m, 0.5H), 4.04-4.15 (m, 2H), 4.24-4.44 (m, 2H), 5.07-5.33 (m, 3.5H), 5.44-5.51 (m, 0.5H), 5.66 (d, 0.5H, J=8.2 Hz), 6.23 (d, 0.5H, J=3.5 Hz).

ESIMS (m/z): 542.2 ([M+Na]⁺), 557.9 ([M+K]⁺).

### (2) Glc-Leu; N-(α/β-D-glucopyranosyloxycarbonyl)-L-leucine

4Ac-Glc-Leu-OMe (300 mg, 0.577 mmol) was dissolved in methanol (6 ml) and water (3 ml), and the mixture was cooled to −10° C. in a thermostatic bath. To this solution was added 1N aqueous lithium hydroxide solution (2.89 ml, 2.89 mmol), and the mixture was stirred for 10 min. To the reaction solution was added water (15 ml), and the mixture was stirred for 20 min. The reaction mixture was treated with a strong acid resin (Amberlite IR-120), and the resin was filtered off. The filtrate was concentrated under reduced pressure to give Glc-Leu (199 mg, yield quant., α:β ratio=1:1) as a white powder.

¹H-NMR (400 MHz, CD₃OD) δ:0.93-1.02 (m, 6H), 1.58-1.85 (m, 3H), 3.34-3.59 (m, 3H), 3.65-3.90 (m, 3H), 4.17-4.25 (m, 1H), 5.35 (d, 0.5H, J=8.0 Hz), 5.96 (d, 0.5H, J=3.8 Hz).

ESIMS (m/z): 360.1 ([M+Na]⁺), 376.1 ([M+K]⁺).

### (3) Glc-Leu-Glc; N—(N-(α/β-D-glucopyranosyloxycarbonyl)-L-leucyl)-β-D-glucopyranosylamine

Glc-Leu (200 mg, 0.59 mmol) was dissolved in tetrahydrofuran (3 ml) at room temperature, and the mixture was cooled in an ice bath. To this solution were added triethylamine (0.119 ml, 1.18 mmol) and pivaloyl chloride (0.085 ml, 0.708 mmol), and the mixture was stirred for 30 min. Then, a solution of D-glucopyranosylamine (137 mg, 0.767 mmol) in methanol/water (2 ml/1 ml) was added. The mixture was warmed to room temperature and stirred for 2 hr. The reaction solution was concentrated under reduced pressure, and a part of the residue was purified by PTLC (dichloromethane/methanol/acetic acid=4/1/0.5) to give Glc-Leu-Glc (6.3 mg, 0.07 mmol, theoretical yield 12%) as a white powder.

¹H-NMR (400 Hz, D₂O) δ: 0.82-0.86 (m, 6H), 1.45-1.66 (m, 3H), 3.29-3.52 (m, 6H), 3.58-3.82 (m, 6H), 4.08-4.14 (m, 1H), 4.87 (d, 0.5H, J=9.1 Hz), 4.88 (d, 0.5H, J=9.1 Hz), 5.31 (d, 0.5H, J=8.1 Hz), 5.88 (d, 0.5H, J=3.5 Hz).

ESIMS (m/z): 521.2 ([M+Na]⁺), 537.2 ([M+K]⁺), 497.1 ([M−H]⁻).

### Example 2

### Phe-Glc; N-(L-phenylalanyl)-β-D-glucopyranosylamine

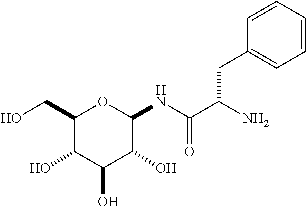 [SMILES: N[C@@H](Cc1ccccc1)C(=O)N[C@@H]1O[C@H](CO)[C@@H](O)[C@H](O)[C@H]1O]

### (1) Z-Phe-Glc; N—(N-benzyloxycarbonyl-L-phenylalanyl)-β-D-glucopyranosylamine

N-benzyloxycarbonyl-L-phenylalanine (Z-Phe) (910 mg, 3.04 mmol) was dissolved in tetrahydrofuran (3 ml) at room temperature, and the mixture was cooled in an ice bath. To this solution were added triethylamine (0.84 ml, 6.0 mmol) and isobutyl chloroformate (0.60 ml, 4.6 mmol) and the mixture was stirred for 30 min. Then, D-glucopyranosylamine (821 mg, 4.6 mmol) dissolved in water (3 ml) was added, and the mixture was warmed to room temperature and stirred for 22 hr. The reaction solution was concentrated under reduced pressure, and the residue was purified by ODS column chromatography (gradient; methanol:water=23:77→58:42) to give Z-Phe-Glc (670 mg, 1.46 mmol, yield 48%) as a white powder.

¹H-NMR (400 MHz, CD₃OD) δ: 2.86 (dd, 1H, J=9.7 Hz, 14.0 Hz), 3.19 (dd, 1H, J=4.6 Hz, 14.0 Hz), 3.26-3.47 (m, 4H), 3.69 (dd, 1H, J=4.7 Hz, 11.9 Hz), 3.86 (dd, 1H, J=2.0 Hz, 10.0 Hz), 4.44 (dd, 1H, J=4.6 Hz, 9.7 Hz), 4.94 (d, 1H, J=9.0 Hz), 4.99 (d, 1H, J=12.5 Hz), 5.05 (d, 1H, J=12.5 Hz), 7.13-7.38 (m, 10H).

ESIMS (m/z): 422.0 ([M+Na]⁺), 821.0 ([2 M+Na]⁺).

### (2) Phe-Glc; N-(L-phenylalanyl)-β-D-glucopyranosylamine

Z-Phe-Glc (251 mg, 0.55 mmol) was dissolved in methanol (8 ml), 2% palladium on carbon catalyst (127 mg) was added, and the mixture was stirred under a hydrogen atmosphere (atmospheric pressure) at room temperature for 40 min. After completion of the reaction, the catalyst was filtered off, and the filtrate was concentrated under reduced pressure to give Phe-Glc (149 mg, 0.46 mmol, yield 84%) as a white powder.

¹H-NMR (400 MHz, CD₃OD) δ: 2.79 (dd, 1H, J=8.1 Hz, 13.6 Hz), 3.11 (dd, 1H, J=5.2 Hz, 13.6 Hz), 3.30-3.47 (m, 4H), 3.59 (dd, 1H, J=5.2 Hz, 8.1 Hz), 3.69 (dd, 1H, J=4.9 Hz, 11.9 Hz), 3.85 (dd, 1H, J=2.1 Hz, 11.9 Hz), 4.93 (d, 1H, J=9.0 Hz), 7.19-7.34 (m, 5H).

ESIMS (m/z): 349.2 ([M+Na]⁺), 365.1 ([M+K]⁺).

### Example 3

### Tyr-Glc; N-(L-tyrosyl)-β-D-glucopyranosylamine

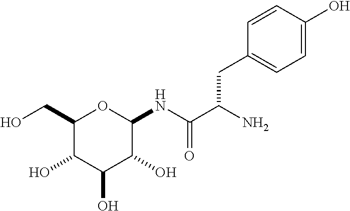 [SMILES: N[C@@H](Cc1ccc(O)cc1)C(=O)N[C@@H]1O[C@H](CO)[C@@H](O)[C@H](O)[C@H]1O]

### (1) Z-Tyr(OBn)-Glc; N—(N-benzyloxycarbonyl-O-benzyl-L-tyrosyl)-β-D-glucopyranosylamine

N-benzyloxycarbonyl-O-benzyl-L-tyrosine (Z-Tyr(OBn)) (3.02 g, 7.48 mmol) was dissolved in tetrahydrofuran (12 ml) at room temperature, and the mixture was cooled in an ice bath. To this solution were added triethylamine (2.1 ml, 15.0 mmol) and isobutyl chloroformate (1.4 ml, 10.8 mmol) and the mixture was stirred for 45 min. Then, D-glucopyranosylamine (2.04 g, 11.3 mmol) dissolved in water (2 ml) and methanol (12 ml) was added. The mixture was warmed to room temperature and stirred for 3 hr, and the reaction solution was concentrated under reduced pressure. The residue was purified by ODS column chromatography (gradient; methanol:water=20:80→58:42) to give Z-Tyr(OBn)-Glc (1.05 g, 1.85 mmol, yield 25%) as a white powder.

¹H-NMR (400 Hz, CD₃OD) δ: 1.28 (dd, 1H, J=9.3 Hz, 13.9 Hz), 1.59 (dd, 1H, J=5.1 Hz, 14.2 Hz), 1.75-1.92 (m, 4H), 2.16 (dd, 1H, J=4.8 Hz, 11.8 Hz), 2.32 (dd, 1H, J=1.7 Hz, 5.6 Hz), 2.86 (dd, 1H, J=4.7 Hz, 9.4 Hz), 3.40 (d, 1H, J=9.0 Hz), 3.46 (d, 1H, J=12.5 Hz), 3.50 (s, 2H), 3.54 (d, 1H, J=12.4 Hz), 5.36-5.39 (m, 1H), 5.37 (d, 1H, J=8.7 Hz), 5.64 (s, 1H), 5.66 (s, 1H), 5.73 (m, 10H).

ESIMS (m/z): 567.1 ([M+H]⁺), 589.2 ([M+Na]⁺), 605.1 ([M+K]⁺), 565.1 ([M−H]⁻).

### (2) Tyr-Glc; N-(L-tyrosyl)-β-D-glucopyranosylamine

Z-Tyr(OBn)-Glc (139 mg, 0.25 mmol) was dissolved in methanol (10 ml) and ethyl acetate (3 ml), 2% palladium on carbon catalyst (71 mg) was added, and the mixture was stirred under a hydrogen atmosphere (atmospheric pressure) at room temperature for 2 hr. After completion of the reaction, the catalyst was filtered off, and the filtrate was concentrated under reduced pressure to give Tyr-Glc (82.3 mg, 0.240 mmol, yield 98%) as a white powder.

¹H-NMR (400 MHz, CD₃OD) δ: 2.71 (dd, 1H, J=7.9 Hz, 13.7 Hz), 3.00 (dd, 1H, J=4.9 Hz, 13.7 Hz), 3.24-3.47 (m, 4H), 3.54 (dd, 1H, J=4.9 Hz, 7.9 Hz), 3.69 (dd, 1H, J=4.9 Hz, 11.9 Hz), 3.86 (dd, 1H, J=2.2 Hz, 11.9 Hz), 4.93 (d, 1H, J=9.0 Hz), 6.74 (d, 1H, J=8.5 Hz), 7.09 (d, 1H, J=8.5 Hz).

ESIMS (m/z): 343.0 ([M+H]⁺), 365.2 ([M+Na]⁺).

### Example 4

### Gly-Glc; N-glycyl-β-D-glucopyranosylamine

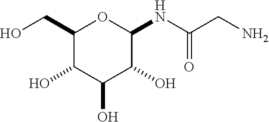 [SMILES: NCC(=O)N[C@@H]1O[C@H](CO)[C@@H](O)[C@H](O)[C@H]1O]

### (1) Z-Gly-Glc; N—(N-(benzyloxycarbonyl)glycyl)-β-D-glucopyranosylamine

N-(benzyloxycarbonyl)glycine (Z-Gly) (546 mg, 2.61 mmol) was dissolved in tetrahydrofuran (4 ml) at room temperature, and the mixture was cooled in an ice bath. To this solution were added triethylamine (0.72 ml, 5.2 mmol) and isobutyl chloroformate (0.50 ml, 3.9 mmol), and the mixture was stirred for 30 min. Then, D-glucopyranosylamine (700 mg, 3.9 mmol) dissolved in water (4 ml) was added, and the mixture was warmed to room temperature and stirred for 21 hr. The reaction solution was concentrated under reduced pressure, and the residue was purified by ODS column chromatography (gradient; methanol:water=19:81→44:56) to give Z-Gly-Glc (382 mg, 1.03 mmol, yield 40%) as a white powder.

¹H-NMR (400 MHz, CD₃OD) δ:3.25-3.45 (m, 4H), 3.66 (dd, 1H, J=5.0 Hz, 11.9 Hz), 3.79-3.85 (m, 1H), 3.85 (d, 2H, J=4.6 Hz), 4.94 (d, 1H, J=9.0 Hz), 5.13 (s, 2H), 7.21-7.40 (m, 5H).

ESIMS (m/z): 393.1 ([M+Na]⁺), 409.0 ([M+K]⁺).

### (2) Gly-Glc; N-glycyl-β-D-glucopyranosylamine

Z-Gly-Glc (245 mg, 0.66 mmol) was dissolved in methanol (3 ml), 2% palladium on carbon catalyst (245 mg) was added and the mixture was stirred under a hydrogen atmosphere (atmospheric pressure) at room temperature for 2 hr. The catalyst was filtered off, and the filtrate was concentrated under reduced pressure. Ethyl acetate (0.5 ml) was added and the mixture was stirred for 3 hr. Filtration gave Gly-Glc (81.5 mg, 0.345 mmol, yield 52%) as a white powder.

¹H-NMR (400 MHz, D₂O) δ: 3.26-3.37 (m, 4H), 3.42-3.50 (m, 2H), 3.64 (dd, 1H, J=5.3 Hz, 12.4 Hz), 3.79 (dd, 1H, J=2.2 Hz, 12.4 Hz), 4.91 (d, 1H, J=9.2 Hz).

ESIMS (m/z): 237.0 ([M+H]⁺), 258.9 ([M+Na]⁺).

### Example 5

### Ala-Glc; N-(L-alanyl)-β-D-glucopyranosylamine

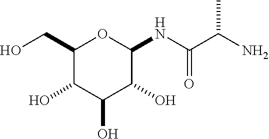 [SMILES: C[C@H](N)C(=O)N[C@@H]1O[C@H](CO)[C@@H](O)[C@H](O)[C@H]1O]

### (1) Z-Ala-Glc; N—(N-benzyloxycarbonyl-L-alanyl)-β-D-glucopyranosylamine

N-benzyloxycarbonyl-L-alanine (Z-Ala) (2.49 g, 11.2 mmol) was dissolved in tetrahydrofuran (18 ml) at room temperature, and the mixture was cooled in an ice bath. To this solution were added triethylamine (3.10 ml, 22.2 mmol) and pivaloyl chloride (1.90 ml, 16.6 mmol) and the mixture was stirred for 30 min. Then, D-glucopyranosylamine (3.04 g, 17.0 mmol) dissolved in water (3 ml) and methanol (18 ml) was added, and the mixture was warmed to room temperature and stirred for 2 hr. The reaction solution was concentrated under reduced pressure, and the residue was purified by ODS column chromatography (gradient; methanol:water=10:90→30:70) to give Z-Ala-Glc (2.94 g, 7.66 mmol, yield 69%) as a white powder.

¹H-NMR (400 MHz, CD₃OD) δ: 1.37 (d, 3H, J=7.2 Hz), 3.26-3.48 (m, 4H), 3.67 (dd, 1H, J=4.8 Hz, 12.0 Hz), 3.81-3.89 (m, 1H), 3.67 (q, 1H, J=7.2 Hz), 4.92 (d, 1H, J=9.0 Hz), 5.09 (d, 1H, J=12.7 Hz), 5.13 (d, 1H, J=12.7 Hz), 7.27-7.45 (m, 5H).

ESIMS (m/z): 385.2 ([M+H]⁺), 402.3 ([M+NH₄]⁺), 407.2 ([M+Na]⁺), 383.2 ([M−H]⁻), 767.3 ([2 M−H]⁻).

### (2) Ala-Glc; N-(L-alanyl)-β-D-glucopyranosylamine

Z-Ala-Glc (132 mg, 0.34 mmol) was dissolved in methanol (3 ml), 2% palladium on carbon catalyst (71 mg) was added and the mixture was stirred under a hydrogen atmosphere (atmospheric pressure) at room temperature for 2 hr. After completion of the reaction, the catalyst was filtered off, and the filtrate was concentrated under reduced pressure to give Ala-Glc (92.9 mg, 0.371 mmol, yield quant.) as a white powder.

¹H-NMR (400 MHz, CD₃OD) δ: 1.30 (d, 3H, J=7.0 Hz), 3.26-3.48 (m, 5H), 3.67 (dd, 1H, J=4.9 Hz, 11.9 Hz), 3.85 (dd, 1H, J=2.0 Hz, 11.9 Hz), 4.91 (d, 1H, J=9.0 Hz).

ESIMS (m/z): 273.1 ([M+Na]⁺).

### Example 6

### Val-Glc; N-(L-valyl)-β-D-glucopyranosylamine

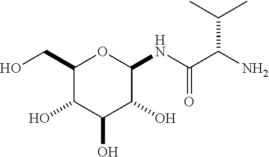 [SMILES: CC(C)[C@H](N)C(=O)N[C@@H]1O[C@H](CO)[C@@H](O)[C@H](O)[C@H]1O]

### (1) Z-Val-Glc; N—(N-benzyloxycarbonyl-L-valyl)-β-D-glucopyranosylamine

N-benzyloxycarbonyl-L-valine (Z-Val) (949 mg, 3.78 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (998 mg, 5.6 mmol) was dissolved in water (6 ml) and added, and the mixture was warmed to room temperature and stirred for 15 hr. The reaction solution was concentrated under reduced pressure, and the residue was purified by ODS column chromatography (gradient; methanol:water=19:81→50:50) to give Z-Val-Glc (1.12 g, 2.7 mmol, yield 72%) as a white powder.

¹H-NMR (400 MHz, CD₃OD) δ: 0.95 (d, 3H, J=6.8 Hz), 1.00 (d, 3H, J=6.8 Hz), 2.02-2.15 (m, 1H), 3.26-3.45 (m, 4H), 3.65-3.71 (m, 1H), 3.79-3.85 (m, 1H), 4.00 (d, 1H, J=6.8 Hz), 4.93 (d, 1H, J=9.0 Hz), 5.09 (d, 1H, J=12.4 Hz), 5.13 (d, 1H, J=12.4 Hz), 7.27-7.51 (m, 5H).

ESIMS (m/z): 237.0 ([M+H]⁺), 258.9 ([M+Na]⁺).

### (2) Val-Glc; N-(L-valyl)-β-D-glucopyranosylamine

Z-Val-Glc (251 mg, 0.608 mmol) was dissolved in methanol (6 ml) and ethyl acetate (0.5 ml), 2% palladium on carbon catalyst (125 mg) was added and the mixture was stirred under a hydrogen atmosphere (atmospheric pressure) at room temperature for 1 hr. After completion of the reaction, the catalyst was filtered off, and the filtrate was concentrated under reduced pressure to give Val-Glc (168 mg, 0.605 mmol, yield quant.) as a white powder.

¹H-NMR (400 MHz, CD₃OD) δ: 0.95 (d, 3H, J=6.9 Hz), 1.00 (d, 3H, J=6.9 Hz), 1.91-2.05 (m, 1H), 3.12 (d, 1H, J=5.8 Hz), 3.24-3.46 (m, 4H), 3.68 (dd, 1H, J=4.7 Hz, 11.9 Hz), 3.84 (dd, 1H, J=1.9 Hz, 11.9 Hz), 4.93 (d, 1H, J=9.0 Hz).

ESIMS (m/z): 279.1 ([M+H]⁺), 301.2 ([M+Na]⁺).

### Example 7

### Leu-Glc; N-(L-leucyl)-β-D-glucopyranosylamine

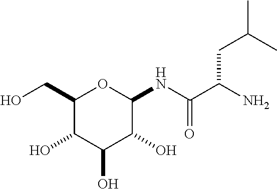 [SMILES: CC(C)C[C@H](N)C(=O)N[C@@H]1O[C@H](CO)[C@@H](O)[C@H](O)[C@H]1O]

### (1) Z-Leu-Glc; N—(N-benzyloxycarbonyl-L-leucyl)-β-D-glucopyranosylamine

N-benzyloxycarbonyl-L-leucine (Z-Leu) (998 mg, 3.76 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (992 mg, 5.5 mmol) dissolved in water (6 ml) was added, and the mixture was warmed to room temperature and stirred for 15 hr. The reaction solution was concentrated under reduced pressure, and the residue was purified by ODS column chromatography (gradient; methanol:water=19:81→47:53) to give Z-Leu-Glc (636 mg, 1.49 mmol, yield 40%) as a white powder.

¹H-NMR (400 MHz, CD₃OD) δ: 0.95 (d, 3H, J=4.4 Hz), 1.00 (d, 3H, J=4.5 Hz), 1.50-1.64 (m, 2H), 1.67-1.79 (m, 1H), 3.34-3.43 (m, 4H), 3.63-3.72 (m, 1H), 3.79-3.87 (m, 1H), 4.21 (dd, 1H, J=5.6 Hz, 9.5 Hz), 4.91 (d, 1H, J=9.0 Hz), 5.09 (d, 1H, J=12.5 Hz), 5.13 (d, 1H, J=12.5 Hz), 7.27-7.41 (m, 5H).

ESIMS (m/z): 449.1 ([M+Na]⁺), 464.9 ([M+k]⁺).

### (2) Leu-Glc; N-(L-leucyl)-β-D-glucopyranosylamine

Z-Leu-Glc (172 mg, 0.402 mmol) was dissolved in methanol (2 ml), 2% palladium on carbon catalyst (91.2 mg) was added, and the mixture was stirred under a hydrogen atmosphere (atmospheric pressure) at room temperature for 1 hr. After completion of the reaction, the catalyst was filtered off, and the filtrate was concentrated under reduced pressure to give Leu-Glc (116 mg, 0.397 mmol, yield 99%) as a white powder.

¹H-NMR (400 MHz, CD₃OD) δ: 0.96 (d, 3H, J=6.6 Hz), 0.97 (d, 3H, J=6.6 Hz), 1.38-1.47 (m, 1H), 1.53-1.61 (m, 1H), 1.69-1.84 (m, 1H), 3.27-3.45 (m, 5H), 3.68 (dd, 1H, J=4.8 Hz, 12.0 Hz), 3.84 (dd, 1H, J=1.9 Hz, 12.0 Hz), 4.92 (d, 1H, J=9.1 Hz).

ESIMS (m/z): 293.2 ([M+H]⁺), 314.9 ([M+Na]⁺).

### Example 8

### Ile-Glc; N-(L-isoleucyl)-β-D-glucopyranosylamine

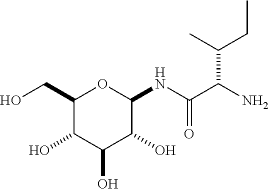 [SMILES: CCC(C)[C@H](N)C(=O)N[C@@H]1O[C@H](CO)[C@@H](O)[C@H](O)[C@H]1O]

### (1) Z-Ile-Glc; N—(N-benzyloxycarbonyl-L-isoleucyl)-β-D-glucopyranosylamine

N-benzyloxycarbonyl-L-isoleucine (Z-Ile) (990 mg, 3.73 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (994 mg, 5.5 mmol) dissolved in water (6 ml) was added, and the mixture was warmed to room temperature and the mixture was stirred for 16 hr. The reaction solution was concentrated under reduced pressure, and the residue was purified by ODS column chromatography (gradient; methanol:water=19:81→50:50) to give Z-Ile-Glc (312 mg, 0.73 mmol, yield 20%) as a white powder.

¹H-NMR (400 MHz, CD₃OD) δ: 0.92 (d, 3H, J=7.4 Hz), 0.97 (dd, 3H, J=2.8 Hz, 6.7 Hz), 1.08-1.27 (m, 1H), 1.50-1.62 (m, 1H), 1.77-1.96 (m, 1H), 3.20-3.44 (m, 4H), 3.64-3.71 (m, 1H), 3.79-3.90 (m, 1H), 4.02 (d, 1H, J=7.4 Hz), 4.92 (d, 1H, J=9.0 Hz), 5.09 (d, 1H, J=12.4 Hz), 5.13 (d, 1H, J=12.4 Hz), 7.26-7.40 (m, 5H).

ESIMS (m/z): 427.0 ([M+H]⁺), 449.0 ([M+Na]⁺), 464.8 ([M+K]⁺), 425.0 ([M−H]⁻).

### (2) Ile-Glc; N-(L-isoleucyl)-β-D-glucopyranosylamine

Z-Ile-Glc (1.94 g, 4.55 mmol) was dissolved in methanol (40 ml) and ethyl acetate (4 ml), 2% palladium on carbon catalyst (934 mg) was added, and the mixture was stirred under a hydrogen atmosphere (atmospheric pressure) at room temperature for 1 hr. After completion of the reaction, the catalyst was filtered off, and the filtrate was concentrated under reduced pressure to give Ile-Glc (1.24 g, 4.25 mmol, yield 93%) as a white powder.

¹H-NMR (400 Hz, D₂O) δ: 0.90 (t, 3H, J=7.41 Hz), 0.97 (d, 3H, J=6.91 Hz), 1.13-1.24 (m, 1H), 1.45-1.53 (m, 1H), 1.77-1.84 (m, 1H), 3.39-3.45 (m, 3H), 3.50-3.54 (m, 1H), 3.55 (t, 1H, J=9.1 Hz), 3.72 (dd, 1H, J=5.3 Hz, 12.4 Hz), 3.88 (dd, 1H, J=2.2 Hz, 12.4 Hz), 5.00 (d, 1H, J=9.2 Hz).

ESIMS (m/z): 292.9 ([M+H]⁺), 315.1 ([M+Na]⁺), 331.0 ([M+K]⁺), 585.1 ([2 M+H]⁺), 607.1 ([2 M+Na]⁺), 290.8 ([M−H]⁻).

### Example 9

### Ser-Glc; N-(L-Seryl)-β-D-glucopyranosylamine

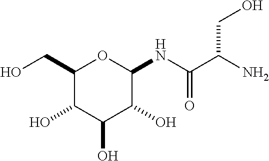 [SMILES: N[C@@H](CO)C(=O)N[C@@H]1O[C@H](CO)[C@@H](O)[C@H](O)[C@H]1O]

### (1) Z-Ser(OBn)-Glc; N—(N-benzyloxycarbonyl-O-benzyl-L-Seryl)-β-D-glucopyranosylamine

N-benzyloxycarbonyl-O-benzyl-L-serine (Z-Ser(OBn)) (1.21 g, 3.67 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (991 mg, 5.5 mmol) was dissolved in water (6 ml) and added, and the mixture was warmed to room temperature and stirred for 16 hr. The reaction solution was concentrated under reduced pressure, and the residue was purified by ODS column chromatography (gradient; methanol:water=19:81→50:50) to give Z-Ser(OBn)-Glc (535 mg, 1.09 mmol, yield 30%) as a white powder.

¹H-NMR (400 MHz, CD₃OD) δ: 3.20-3.49 (m, 4H), 3.68 (dd, 1H, J=4.8 Hz, 11.9 Hz), 3.75 (d, 2H, J=5.5 Hz), 3.84 (dd, 1H, J=2.0 Hz, 11.9 Hz), 4.44 (t, 1H, J=5.5 Hz), 4.56 (s, 2H), 4.94 (d, 1H, J=9.0 Hz), 5.10 (d, 1H, J=12.3 Hz), 5.15 (d, 1H, J=12.3 Hz), 7.22-7.41 (m, 4H).

ESIMS (m/z): 513.1 ([M+Na]⁺), 529.0 ([M+K]⁺).

### (2) Ser-Glc; N-(L-Seryl)-β-D-glucopyranosylamine

In the same manner as in Example 8, step (2), Ser-Glc (61.8 mg, 0.232 mmol, yield 48%) was obtained from Z-Ser(OBn)-Glc (221.4 mg, 0.480 mmol) as a white powder.

¹H-NMR (400 MHz, D₂O) δ: 3.29-3.38 (m, 2H), 3.41-3.50 (m, 2H), 3.56 (t, 1H, J=5.0 Hz), 3.62 (dd, 1H, J=5.5 Hz, 12.3 Hz), 3.68-3.75 (m, 2H), 3.79 (dd, 1H, J=2.1 Hz, 12.3 Hz), 4.93 (d, 1H, J=9.2 Hz).

ESIMS (m/z): 267.1 ([M+H]⁺), 289.1 ([M+Na]⁺), 533.2 ([2 M+H]⁺), 265.0 ([M−H]⁻).

### Example 10

### Lys-Glc; N-(L-lysyl)-β-D-glucopyranosylamine

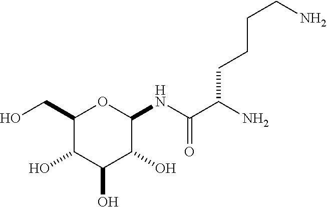 [SMILES: NCCCC[C@H](N)C(=O)N[C@@H]1O[C@H](CO)[C@@H](O)[C@H](O)[C@H]1O]

### (1) Z-Lys(Z)-Glc; N—(N2,N6-bis(benzyloxycarbonyl)-L-lysyl)-β-D-glucopyranosylamine

N2,N6-bis(benzyloxycarbonyl)-L-lysine(Z-Lys(Z)) (1.52 g, 3.66 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.04 g, 5.8 mmol) dissolved in water (6 ml) was added, and the mixture was warmed to room temperature and stirred for 16 hr. The reaction solution was concentrated under reduced pressure, and the residue was purified by ODS column chromatography (gradient; methanol:water=19:81→47:53) to give Z-Lys(Z)-Glc (893 mg, 1.55 mmol, yield 42%) as a white powder.

¹H-NMR (400 MHz, CD₃OD) δ: 1.35-1.58 (m, 4H), 1.61-1.72 (m, 1H), 1.74-1.87 (m, 1H), 3.13 (t, 2H, J=6.8 Hz), 3.63-3.70 (m, 1H), 3.79-3.86 (m, 1H), 4.13 (dd, 1H, J=4.8 Hz, 9.3 Hz), 4.91 (d, 1H, J=8.9 Hz), 5.05-5.14 (m, 4H), 7.23-7.42 (m, 10H).

ESIMS (m/z): 576.2 ([M+H]⁺), 598.1 ([M+Na]⁺), 614.1 ([M+K]⁺).

### (2) Lys-Glc; N-(L-lysyl)-β-D-glucopyranosylamine

Z-Lys(Z)-Glc (199 mg, 0.35 mmol) was dissolved in methanol (5 ml), 20% palladium hydroxide on carbon catalyst (101 mg) was added, and the mixture was stirred under a hydrogen atmosphere (atmospheric pressure) at room temperature for 2 hr. The catalyst was filtered off, 20% palladium hydroxide on carbon catalyst (99.2 mg) was added to the filtrate, and the mixture was stirred under a hydrogen atmosphere (atmospheric pressure) at room temperature for 2 hr. After completion of the reaction, the catalyst was filtered off, and the filtrate was concentrated under reduced pressure to give Lys-Glc (95.2 mg, 0.31 mmol, yield 90%) as a white powder.

¹H-NMR (400 MHz, CD₃OD) δ: 1.48-1.69 (m, 6H), 2.72 (t, 2H, J=7.1 Hz), 3.25-3.48 (m, 5H), 3.67 (dd, 1H, J=5.0 Hz, 11.9 Hz), 3.85 (dd, 1H, J=2.1 Hz, 11.9 Hz), 4.93 (d, 1H, J=9.1 Hz).

ESIMS (m/z): 308.0 ([M+H]⁺), 330.2 ([M+Na]⁺), 615.4 ([2 M+H]⁺), 306.3 ([M+H]⁺), 306.3 ([M−H]⁻), 342.3 ([M−Cl]⁻), 613.4 ([2 M−H]⁻).

### Example 11

### Pro-Glc; N-(L-prolyl)-β-D-glucopyranosylamine

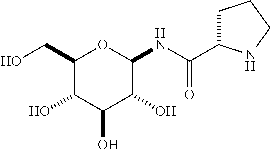 [SMILES: O=C(N[C@@H]1O[C@H](CO)[C@@H](O)[C@H](O)[C@H]1O)[C@@H]1CCCN1]

### (1) Z-Pro-Glc; N—(N-benzyloxycarbonyl-L-prolyl)-β-D-glucopyranosylamine

N-benzyloxycarbonyl-L-proline (Z-Pro) (919 mg, 3.69 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.02 g, 5.7 mmol) dissolved in water (6 ml) was added, and the mixture was warmed to room temperature and stirred for 16 hr. The reaction solution was concentrated under reduced pressure, and the residue was purified by ODS column chromatography (gradient; methanol:water=40:60→64:36) to give Z-Pro-Glc (721 mg, 1.76 mmol, yield 48%) as a white powder.

¹H-NMR (400 MHz, CD₃OD) δ: 1.83-2.11 (m, 3H), 2.15-2.34 (m, 1H), 3.25-3.72 (m, 7H), 3.80-3.88 (m, 1H), 4.28-4.38 (m, 1H), 4.93 (d, 1H, J=9.0 Hz), 5.07-5.19 (m, 1H), 7.22-7.45 (m, 5H).

ESIMS (m/z): 432.9 ([M+Na]⁺), 449.1 ([M+K]⁺).

### (2) Pro-Glc; N-(L-prolyl)-β-D-glucopyranosylamine

Z-Pro-Glc (199 mg, 0.484 mmol) was dissolved in methanol (3 ml), 2% palladium on carbon catalyst (100 mg) was added and the mixture was stirred under a hydrogen atmosphere (atmospheric pressure) at room temperature for 3 hr. The catalyst was filtered off, and the filtrate was concentrated under reduced pressure and dissolved in methanol (3 ml). 2% Palladium on carbon catalyst (96.4 mg) was added and the mixture was stirred under a hydrogen atmosphere (atmospheric pressure) at room temperature for 15 hr. After completion of the reaction, the catalyst was filtered off, and the filtrate was concentrated under reduced pressure to give Pro-Glc (133 mg, 0.48 mmol, yield quant.) as a white powder.

¹H-NMR (400 MHz, CD₃OD) δ: 1.72-1.81 (m, 3H), 2.09-2.19 (m, 1H), 2.89-2.97 (m, 1H), 2.99-3.06 (m, 1H), 3.25-3.45 (m, 4H), 3.64-3.72 (m, 2H), 3.84 (dd, 1H, J=2.1 Hz, 12.0 Hz), 4.89 (d, 1H, J=9.5 Hz).

ESIMS (m/z): 277.3 ([M+H]⁺), 299.3 ([M+Na]⁺), 553.3 ([2 M+H]⁺), 575.3 ([2 M+Na]⁺), 275.3 ([M−H]⁻), 311.1 ([M+Cl]⁻), 551.3 ([2 M−H]⁻).

### Example 12

### Thr-Glc; N-(L-threonyl)-β-D-glucopyranosylamine

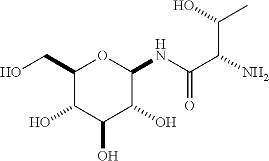 [SMILES: C[C@@H](O)[C@H](N)C(=O)N[C@@H]1O[C@H](CO)[C@@H](O)[C@H](O)[C@H]1O]

### (1) Z-Thr(OBn)-Glc; N—(N-benzyloxycarbonyl-O-benzyl-L-threonyl)-β-D-glucopyranosylamine

N-benzyloxycarbonyl-O-benzyl-L-threonine (Z-Thr(OBn)) (1.28 g, 3.74 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.00 g, 5.6 mmol) dissolved in water (6 ml) was added, and the mixture was warmed to room temperature and stirred for 21 hr. The reaction solution was concentrated under reduced pressure, and the residue was purified by ODS column chromatography (gradient; methanol:water=19:81→47:53) to give Z-Thr(OBn)-Glc (1.28 g, 2.53 mmol, yield 68%) as a white powder.

¹H-NMR (400 Hz, CD₃OD) δ: 1.18 (t, 1H, J=7.0 Hz), 1.19 (s, 1H), 1.20 (s, 1H), 3.42 (t, 1H, J=8.9 Hz), 3.49 (dd, 1H, J=7.0 Hz, 14.0 Hz), 3.65-3.69 (m, 1H), 3.80 (dd, 1H, J=1.7 Hz, 12.0 Hz), 4.06-4.08 (m, 1H), 4.25 (d, 1H, J=3.5 Hz), 4.46-4.61 (m, 1H), 4.54 (d, 1H, J=5.3 Hz), 4.95 (d, 1H, J=9.0 Hz), 5.09 (d, 1H, J=12.4 Hz), 5.14 (d, 1H, J=12.4 Hz), 7.22-7.38 (m, 10H).

ESIMS (m/z): 567.4 ([M+H]⁺), 589.3 ([M+Na]⁺), 565.2 ([M−H]⁻).

### (2) Thr-Glc; N-(L-threonyl)-β-D-glucopyranosylamine

Z-Thr(OBn)-Glc (102 mg, 0.20 mmol) was dissolved in methanol (4 ml), 20% palladium hydroxide on carbon catalyst (108 mg) was added and the mixture was stirred under a hydrogen atmosphere (atmospheric pressure) at room temperature for 3 hr. The catalyst was filtered off, and the filtrate was concentrated under reduced pressure and dissolved in methanol (4 ml). 20% Palladium hydroxide on carbon catalyst (61 mg) was added and the mixture was stirred under a hydrogen atmosphere (atmospheric pressure) at room temperature for 1 hr. Then, 20% palladium hydroxide on carbon catalyst (74 mg) was added and the mixture was stirred under a hydrogen atmosphere (atmospheric pressure) at room temperature for 15 hr. After completion of the reaction, the catalyst was filtered off, and the filtrate was concentrated under reduced pressure to give Thr-Glc (50.6 mg, 0.18 mmol, yield 90%) as a white powder.

¹H-NMR (400 MHz, CD₃OD) δ: 1.26 (d, 3H, J=6.4 Hz), 3.26-3.48 (m, 5H), 3.66 (dd, 1H, J=5.2 Hz, 11.9 Hz), 3.85 (dd, 1H, J=2.1 Hz, 11.9 Hz), 4.01-4.09 (m, 1H), 4.95 (d, 1H, J=9.0 Hz).

ESIMS (m/z): 281.0 ([M+H]⁺), 303.1 ([M+Na]⁺).

### Example 13

### Met-Glc; N-(L-methionyl)-β-D-glucopyranosylamine

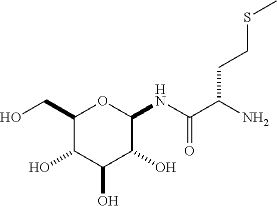 [SMILES: CSCC[C@H](N)C(=O)N[C@@H]1O[C@H](CO)[C@@H](O)[C@H](O)[C@H]1O]

### (1) Fmoc-Met-Glc; N—(N-(9-fluorenylmethyloxycarbonyl)-L-methionyl)-β-D-glucopyranosylamine

N-(9-fluorenylmethyloxycarbonyl)-L-methionine (Fmoc-Met) (1.38 g, 3.70 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.03 g, 5.7 mmol) dissolved in water (1 ml) and methanol (9 ml) was added, and the mixture was warmed to room temperature and stirred for 1.5 hr. The reaction solution was concentrated under reduced pressure, and the residue was purified by ODS column chromatography (gradient; methanol:water=23:77→58:42) to give Fmoc-Met-Glc (531 mg, 1.00 mmol, yield 27%) as a white powder.

¹H-NMR (400 Hz, DMSO-d₄) δ: 1.73-1.82 (m, 1H), 1.85-1.94 (m, 1H), 2.03 (s, 3H), 2.37-2.46 (m, 2H), 3.02-3.12 (m, 3H), 2.37-2.46 (m, 2H), 3.61-3.65 (m, 1H), 4.08-4.16 (m, 1H), 4.20-4.33 (m, 3H), 4.47 (t, 1H, J=5.6 Hz), 4.69 (t, 1H, J=8.8 Hz), 4.81 (d, 1H, J=5.6 Hz), 4.88 (d, 1H, J=5.0 Hz), 4.98 (d, 1H, J=4.7 Hz), 7.27-7.36 (m, 3H), 7.38-7.43 (m, 3H), 7.38-7.43 (m, 2H), 7.48 (d, 1H, J=8.7 Hz), 7.66 (d, 1H, J=6.9 Hz), 7.73 (t, 2H, J=7.9 Hz), 7.85 (d, 1H, J=7.6 Hz), 7.88 (s, 1H), 7.90 (s, 1H), 8.41 (d, 1H, J=8.8 Hz).

ESIMS (m/z): 555.0 ([M+Na]⁺).

### (2) Met-Glc; N-(L-methionyl)-β-D-glucopyranosylamine

To Fmoc-Met-Glc (49.4 mg, 0.16 mmol) was added a solution (1 ml) of 20% piperidine in N,N-dimethylformamide under ice-cooling, and the mixture was stirred at room temperature for 2 hr. After completion of the reaction, the residue was purified by ODS column chromatography (gradient; methanol:water=0:100→40:60) to give Met-Glc (19.0 mg, 0.061 mmol, yield 38%) as a pale-yellow powder.

¹H-NMR (400 Hz, CD₃OD) δ: 2.05-2.16 (m, 1H), 2.23-2.36 (m, 1H), 2.40 (s, 3H), 2.83-2.92 (m, 2H), 3.57-3.65 (m, 1H), 3.66-3.72 (m, 2H), 3.74-3.79 (m, 2H), 3.97 (dd, 1H, J=4.8 Hz, 11.9 Hz), 4.14 (dd, 1H, 1.90, 11.8), 5.22 (d, 1H, J=9.0 Hz).

ESIMS (m/z): 310.8 ([M+H]⁺), 333.0 ([M+Na]⁺).

### Example 14

### Glu-Glc; N-(L-α-glutamyl)-β-D-glucopyranosylamine

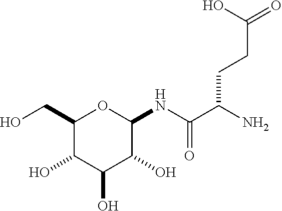 [SMILES: N[C@@H](CCC(=O)O)C(=O)N[C@@H]1O[C@H](CO)[C@@H](O)[C@H](O)[C@H]1O]

### (1) Z-Glu(OBn)-Glc; benzyl (4S)-4-(benzyloxycarbonylamino)-4-(β-D-glucopyranosylaminocarbonyl)butyrate

δ-Benzyl N-benzyloxycarbonyl-L-glutamate (Z-Glu(OBn)) (1.38 g, 3.71 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.00 g, 5.6 mmol) dissolved in water (1 ml) and methanol (6 ml) was added, and the mixture was warmed to room temperature and stirred for 1.5 hr. The reaction solution was concentrated under reduced pressure, and the residue was purified by ODS column chromatography (gradient; methanol:water=23:77→58:42) to give Z-Glu(OBn)-Glc (631 mg, 1.19 mmol, yield 32%) as a white powder.

¹H-NMR (400 Hz, CD₃OD) δ: 1.87 (m, 1H), 2.07-2.18 (m, 1H), 2.48 (t, 2H, J=7.6 Hz), 3.19-3.44 (m, 3H), 3.54 (t, 1H, J=6.6 Hz), 3.64 (dd, 1H, J=4.8 Hz, 11.9 Hz), 3.81 (dd, 1H, J=1.8 Hz, 11.3 Hz), 4.16-4.21 (m, 1H), 4.90 (d, 1H, J=9.0 Hz), 5.08 (d, 2H, J=4.6 Hz), 5.10 (s, 2H), 7.26-7.36 (m, 10H).

ESIMS (m/z): 554.9 ([M+Na]⁺), 571.0 ([M+K]⁺).

### (2) Glu-Glc; N-(L-α-glutamyl)-β-D-glucopyranosylamine

Z-Glu(OBn)-Glc (31.6 mg, 0.059 mmol) was dissolved in methanol (1 ml), 2% palladium on carbon catalyst (20.0 mg) was added and the mixture was stirred under a hydrogen atmosphere (atmospheric pressure) at room temperature for 1 hr. The catalyst was filtered off, and the filtrate was concentrated under reduced pressure and dissolved in a mixed solvent of methanol (1 ml) and water (glass pipette 7 drops). 2% Palladium on carbon catalyst (16.7 mg) was added and the mixture was stirred under a hydrogen atmosphere (atmospheric pressure) at room temperature for 24 hr. After completion of the reaction, the catalyst was filtered off, and the filtrate was concentrated under reduced pressure to give Glu-Glc (12.3 mg, 0.039 mmol, yield 67%) as a white powder.

¹H-NMR (400 Hz, D₂O) δ: 2.06-2.23 (m, 2H), 2.39 (t, 2H, J=7.4 Hz), 3.42 (t, 2H, J=9.4 Hz), 3.50-3.58 (m, 2H), 3.71 (dd, 1H, J=5.1 Hz, 12.4 Hz), 3.87 (dd, 1H, J=2.2 Hz, 12.4 Hz), 4.08 (dd, 1H, J=5.3 Hz, 7.5 Hz), 5.01 (m, 1H).

ESIMS (m/z): 331.0 ([M+Na]⁺).

### Example 15

### Cys-Glc hydrochloride; N-(L-cysteinyl)-β-D-glucopyranosylamine hydrochloride

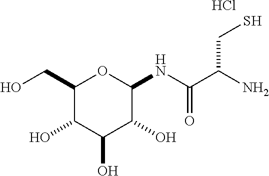 [SMILES: Cl ; N[C@@H](CS)C(=O)N[C@@H]1O[C@H](CO)[C@@H](O)[C@H](O)[C@H]1O]

### (1) Boc-Cys(Trt)-Glc; N—(N-tert-butyloxycarbonyl-S-trityl-L-cysteinyl)-β-D-glucopyranosylamine

N-tert-butyloxycarbonyl-S-trityl-L-cysteine (Boc-Cys(Trt)) (3.51 g, 7.56 mmol) was dissolved in tetrahydrofuran (12 ml) at room temperature, and the mixture was cooled in an ice bath. To this solution were added triethylamine (2.08 ml, 14.9 mmol) and isobutyl chloroformate (1.45 ml, 11.2 mmol) and the mixture was stirred for 50 min. Then, D-glucopyranosylamine (2.00 g, 11.2 mmol) dissolved in water (3 ml) and methanol (18 ml) was added, and the mixture was warmed to room temperature and stirred for 1.5 hr. The reaction solution was concentrated under reduced pressure, and the residue was purified by ODS column chromatography (gradient; methanol:water=23:77→73:27) to give Boc-Cys(Trt)-Glc (991 mg, 1.59 mmol, yield 21%) as a pale-yellow powder.

¹H-NMR (400 MHz, CD₃OD) δ: 1.46 (s, 9H), 3.21-3.43 (m, 4H), 3.61-3.69 (m, 1H), 3.78-3.85 (m, 1H), 3.94-4.08 (m, 1H), 4.83 (d, 1H, J=9.0 Hz), 7.18-7.46 (m, 15H).

ESIMS (m/z): 623.2 ([M−H]⁻).

### (2) Cys-Glc hydrochloride; N-(L-cysteinyl)-β-D-glucopyranosylamine hydrochloride

To Boc-Cys(Trt)-Glc (300 mg, 0.48 mmol) was added a solution (10 ml) of 4N hydrogen chloride in dioxane under ice-cooling, and the mixture was stirred at room temperature for 2 hr. The reaction solution was concentrated, and the obtained residue was purified by ODS column chromatography (gradient; methanol:water=0:100→15:85) to give Cys-Glc hydrochloride (126 mg, 0.316 mmol, yield 83%) as a pale-yellow powder.

¹H-NMR (400 MHz, CD₃OD) δ: 3.01 (dd, 1H, J=7.0 Hz, 14.8 Hz), 3.10 (dd, 1H, J=4.5 Hz, 14.8 Hz), 3.23-3.46 (m, 4H), 3.68 (dd, 1H, J=5.0 Hz, 11.9 Hz), 3.85 (dd, 1H, J=2.0 Hz, 11.9 Hz), 4.06 (dd, 1H, J=4.5 Hz, 7.0 Hz), 4.97 (d, 1H, J=9.1 Hz).

ESIMS (m/z): 317.1 ([M−H]⁻).

### Example 16

### Asp-Glc; N-(L-α-aspartyl)-β-D-glucopyranosylamine

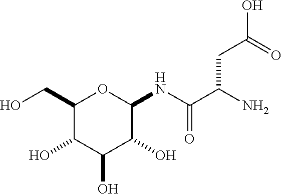 [SMILES: N[C@@H](CC(=O)O)C(=O)N[C@@H]1O[C@H](CO)[C@@H](O)[C@H](O)[C@H]1O]

### (1) Z-Asp(OBn)-Glc; benzyl (3S)-3-(benzyloxycarbonylamino)-3-(β-D-glucopyranosylaminocarbonyl)propionate

γ-Benzyl N-benzyloxycarbonyl-L-aspartate (Z-Asp(OBn)) (1.35 g, 3.78 mmol) was dissolved in tetrahydrofuran (6 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (998 mg, 5.6 mmol) dissolved in water (1 ml) and methanol (8 ml) was added, and the mixture was warmed to room temperature and stirred for 2 hr. The reaction solution was concentrated under reduced pressure, water (15 ml) and methanol (1 ml) were added to the residue, and the mixture was extracted 5 times with dichloromethane. The organic layer was washed with 15% brine (50 ml), and dried over magnesium sulfate. The desiccant was filtered off, and the filtrate was concentrated under reduced pressure. The residue was purified by silica gel column chromatography (gradient; methanol:ethyl acetate=1:99→9:91) to give Z-Asp(OBn)-Glc (67.2 mg, 0.130 mmol, yield 3%) as a white powder.

¹H-NMR (400 Hz, CD₃OD) δ: 2.74 (dd, 1H, J=8.6 Hz, 16.2 Hz), 2.92 (dd, 1H, J=5.1 Hz, 16.3 Hz), 3.27-3.41 (m, 3H), 3.62-3.67 (m, 1H), 3.80 (dd, 1H, J=11.2 Hz), 3.92 (dd, 1H, J=6.5 Hz), 4.60-4.66 (m, 1H), 4.88 (d, 1H, J=9.1 Hz), 5.09 (d, 2H, J=7.0 Hz), 5.12 (s, 2H), 7.26-7.40 (m, 10H).

ESIMS (m/z): 540.9 ([M+Na]⁺), 556.8 ([M+K]⁺).

### (2) Asp-Glc; N-(L-α-aspartyl)-β-D-glucopyranosylamine

Z-Asp(OBn)-Glc (61.3 mg, 0.118 mmol) was dissolved in methanol (4 ml), 20% palladium hydroxide on carbon catalyst (30.2 mg) was added and the mixture was stirred under a hydrogen atmosphere (atmospheric pressure) at room temperature for 5 hr. After argon substitution, 20% palladium hydroxide on carbon catalyst (29.5 mg) was further added, and the mixture was stirred under a hydrogen atmosphere (atmospheric pressure) at room temperature for 16 hr. After completion of the reaction, the catalyst was filtered off, and the filtrate was concentrated under reduced pressure to give Asp-Glc (25.8 mg, 0.088 mmol, yield 74%) as a white powder.

¹H-NMR (400 Hz, D₂O) δ: 2.78 (dd, 1H, J=8.5 Hz, 17.5 Hz), 2.90 (dd, 1H, J=4.8 Hz, 17.5 Hz), 3.42 (t, 2H, J=9.1 Hz), 3.50-3.54 (m, 1H), 3.55 (t, 1H, J=9.1 Hz), 3.71 (dd, 1H, J=5.3 Hz, 12.4 Hz), 3.87 (dd, 1H, J=2.1 Hz, 12.3 Hz), 4.30 (dd, 1H, J=4.8 Hz, 8.5 Hz), 5.01 (d, 1H, J=9.1 Hz).

ESIMS (m/z): 294.9 ([M+H]⁺), 317.0 ([M+Na]⁺), 333.0 ([M+K]⁺), 292.8. ([M−H]⁻), 587.0 ([2 M−H]⁻).

### Example 17

### Gln-Glc; N-(L-glutaminyl)-β-D-glucopyranosylamine

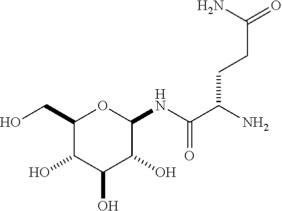 [SMILES: NC(=O)CC[C@H](N)C(=O)N[C@@H]1O[C@H](CO)[C@@H](O)[C@H](O)[C@H]1O]

### (1) Z-Gln-Glc; N—(N-benzyloxycarbonyl-L-glutaminyl)-β-D-glucopyranosylamine

N-benzyloxycarbonyl-L-glutamine (Z-Gln) (1.05 g, 3.76 mmol) was dissolved in tetrahydrofuran (6 ml) and N-methylpyrrolidone (3.5 ml) at room temperature, and the mixture was cooled in an ice bath. To this solution were added triethylamine (1.04 ml, 7.5 mmol) and isobutyl chloroformate (0.72 ml, 5.6 mmol) and the mixture was stirred for 30 min. Then, D-glucopyranosylamine (1.04 g, 5.8 mmol) dissolved in water (1 ml) and methanol (8 ml) was added, and the mixture was warmed to room temperature and stirred for 2 hr. The reaction solution was concentrated under reduced pressure, and the residue was purified by ODS column chromatography (gradient; methanol:water=0:100→30:70) to give Z-Gln-Glc (685 mg, 1.55 mmol, yield 41%) as a white powder.

¹H-NMR (400 Hz, CD₃OD) δ: 1.88-1.96 (m, 1H), 2.04-2.12 (m, 1H), 3.27-3.24 (m, 3H), 3.64 (dd, 1H, J=4.8 Hz, 11.9 Hz), 3.82 (dd, 1H, J=1.8 Hz, 11.9 Hz), 3.94 (dd, 1H, J=4.7 Hz, 6.6 Hz), 4.14-4.18 (m, 1H), 4.90 (d, 1H, J=8.9 Hz), 5.09 (s, 2H), 7.27-7.46 (m, 5H).

ESIMS (m/z): 463.9 ([M+Na]⁺), 480.0 ([M+K]⁺).

### (2) Gln-Glc; N-(L-glutaminyl)-β-D-glucopyranosylamine

Z-Gln-Glc (30.2 mg, 0.068 mmol) was dissolved in methanol (4 ml), 2% palladium on carbon catalyst (19.9 mg) was added and the mixture was stirred under a hydrogen atmosphere (atmospheric pressure) at room temperature for 2 hr. The catalyst was filtered off, and the filtrate was concentrated under reduced pressure and dissolved in methanol (4 ml). 2% Palladium on carbon catalyst (17.9 mg) was added and the mixture was stirred under a hydrogen atmosphere (atmospheric pressure) at room temperature for 6 hr. After completion of the reaction, the catalyst was filtered off, and the filtrate was concentrated under reduced pressure to give Gln-Glc (13.0 mg, 0.042 mmol, yield 62%) as a white powder.

¹H-NMR (400 Hz, CD₃OD) δ: 1.85-1.91 (m, 1H), 1.95-2.02 (m, 1H), 3.25-3.44 (m, 4H), 3.64 (dd, 1H, J=5.1 Hz, 11.9 Hz), 3.79 (d, 1H, J=6.9 Hz), 3.83 (dd, 1H, J=2.0 Hz, 11.9 Hz), 4.91 (d, 1H, J=9.1 Hz).

ESIMS (m/z): 307.9 ([M+H]⁺), 330.1 ([M+Na]⁺).

### Example 18

### Trp-Glc; N-(L-tryptophyl)-β-D-glucopyranosylamine

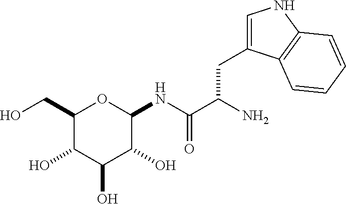 [SMILES: N[C@@H](Cc1c[nH]c2ccccc12)C(=O)N[C@@H]1O[C@H](CO)[C@@H](O)[C@H](O)[C@H]1O]

### (1) Boc-Trp(Boc)-Glc; N—(N,N′-di-tert-butyloxycarbonyl-L-tryptophyl)-β-D-glucopyranosylamine

N,N′-di-tert-butyloxycarbonyl-L-tryptophan (Boc-Trp(Boc)) (704 mg, 1.74 mmol) was dissolved in tetrahydrofuran (3 ml) at room temperature, and the mixture was cooled in an ice bath. To this solution were added triethylamine (0.35 ml, 2.61 mmol) and isobutyl chloroformate (0.35 ml, 2.62 mmol) and the mixture was stirred for 30 min. Then, D-glucopyranosylamine (463 mg, 2.61 mmol) dissolved in methanol/water (4 ml/1 ml) was added. The mixture was warmed to room temperature and stirred for 1.5 hr. The reaction solution was concentrated under reduced pressure, and the residue was purified by ODS column chromatography (gradient; methanol:water=23:77→58:42) to give Boc-Trp(Boc)-Glc (193 mg, 0.34 mmol, yield 20%) as a pale-yellow powder.

¹H-NMR (400 MHz, CD₃OD) δ: 1.36 (s, 9H), 1.69 (s, 9H), 2.95-3.00 (m, 1H), 3.25-3.44 (m, 3H), 3.69-3.73 (m, 1H), 3.85-3.88 (m, 1H), 4.45 (dd, 1H, J=4.6 Hz, 9.3 Hz), 4.96 (d, 1H, J=9.1 Hz), 7.24-7.33 (m, 2H), 7.53 (s, 1H), 7.68 (d, 1H, J=7.5 Hz), 8.10 (d, 1H, J=8.2 Hz).

ESIMS (m/z): 588.1 ([M+Na]⁺), 603.9 ([M+K]⁺), 564.0 ([M−H]⁻).

### (2) Trp-Glc; N-(L-tryptophyl)-β-D-glucopyranosylamine

Boc-Trp(Boc)-Glc (30.5 mg, 0.05 mmol) was cooled in an ice bath, 4N hydrogen chloride/dioxane (4 ml) was added and the mixture was warmed to room temperature and stirred for 50 min. The reaction mixture was concentrated under reduced pressure, dissolved in methanol/water (1 ml/1 ml), neutralized with Amberlite-OH resin, and the resin was filtered off. The residue was concentrated to give Trp-Glc (8.0 mg, 0.022 mmol, yield 44%) as a pale-yellow powder.

¹H-NMR (400 MHz, D₂O) δ: 3.02-3.14 (2H, m), 3.24-3.46 (m, 4H), 3.64 (dd, 1H, J=4.9 Hz, 13.5 Hz), 3.70 (t, 1H, J=6.3 Hz), 3.78 (dd, 1H, J=2.4 Hz, 12.3 Hz), 7.07 (dt, 2H, J=0.9 Hz, 7.9 Hz), 7.15 (dt, 1H, J=1.0 Hz, 8.1 Hz), 7.15 (s, 1H), 7.41 (d, 1H, J=8.2 Hz), 7.61 (d, 1H, J=7.8 Hz).

ESIMS (m/z): 366.1 ([M+H]⁺), 388.1 ([M+Na]⁺), 731.1 ([2 M+H]⁺), 363.7 ([M−H]⁻).

### Example 19

### His-Glc; N-(L-histidyl)-β-D-glucopyranosylamine

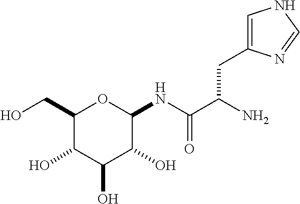 [SMILES: N[C@@H](Cc1c[nH]cn1)C(=O)N[C@@H]1O[C@H](CO)[C@@H](O)[C@H](O)[C@H]1O]

### (1) Z-His(Z)-Glc; N—(N,N′-bis(benzyloxycarbonyl)-L-histidyl)-β-D-glucopyranosylamine

In the same manner as in Example 2, step (1), Z-His(Z)-Glc (49.7 mg, 0.085 mmol, yield 6%) was obtained as a pale-yellow powder from N,N′-bis(benzyloxycarbonyl)-L-histidine (Z-His(Z)) (715 mg, 1.49 mmol).

¹H-NMR (400 MHz, CD₃OD) δ: 2.87-2.99 (1H, m), 3.01-3.16 (1H, m), 3.31-3.42 (3H, m), 3.66-3.77 (1H, m), 3.81-3.89 (2H, m), 4.20-4.92 (1H, m), 4.98-5.19 (3H, m), 5.43 (2H, d, J=5.9 Hz), 7.14-7.50 (11H, m), 8.81 (1H, s).

ESIMS (m/z): 585.0 ([M+H]⁺), 606.9 ([M+Na]⁺), 583.1 ([M−H]⁻).

### (2) His-Glc; N-(L-histidyl)-β-D-glucopyranosylamine

Z-His(Z)-Glc (21.6 mg, 0.035 mmol) was dissolved in methanol (1 ml), 2% palladium on carbon catalyst (24.3 mg) was added and the mixture was stirred under a hydrogen atmosphere (atmospheric pressure) at room temperature for 1.5 hr. The catalyst was filtered off, and the filtrate was concentrated under reduced pressure and ¹H-NMR was measured to confirm residual of Z group. The residue was dissolved again in methanol (1 ml), 20% palladium hydroxide on carbon catalyst (18.2 mg) was added and the mixture was stirred under a hydrogen atmosphere (atmospheric pressure) at room temperature for 1.5 hr. The catalyst was filtered off, and the filtrate was concentrated under reduced pressure and ¹H-NMR was measured to confirm residual of Z group. The residue was dissolved in methanol (1 ml) and water (glass pipette several drops), 20% palladium hydroxide on carbon catalyst (18.2 mg) was added and the mixture was stirred under a hydrogen atmosphere (atmospheric pressure) at room temperature for 1.5 hr. After completion of the reaction, the catalyst was filtered off, and the filtrate was concentrated under reduced pressure to give His-Glc (8.6 mg, 0.027 mmol, yield 77%) as a pale-yellow powder.

¹H-NMR (400 MHz, D₂O) δ: 2.74-2.92 (m, 1H), 3.25-3.50 (m, 3H), 3.61-3.68 (m, 2H), 3.71-3.80 (m, 2H), 4.86 (d, 1H, J=9.1 Hz), 6.88 (s, 1H), 7.59 (s, 1H).

ESIMS (m/z): 317.0 ([M+H]⁺), 339.0 ([M+Na]⁺), 314.7 ([M−H]⁻).

### Example 20

### Arg-Glc; N-(L-arginyl)-β-D-glucopyranosylamine

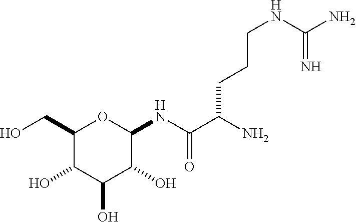 [SMILES: N=C(N)NCCC[C@H](N)C(=O)N[C@@H]1O[C@H](CO)[C@@H](O)[C@H](O)[C@H]1O]

### (1) Z-Arg(Z)₂-Glc; N-(tris(benzyloxycarbonyl)-L-arginyl)-β-D-glucopyranosylamine

tris(benzyloxycarbonyl)-L-arginine (Z-Arg(Z)₂) (710 mg, 1.21 mmol) was dissolved in tetrahydrofuran (5 ml) at room temperature, and the mixture was cooled in an ice bath. To this solution were added triethylamine (0.34 ml, 2.42 mmol) and isobutyl chloroformate (0.24 ml, 1.82 mmol) and the mixture was stirred for 30 min. Then, D-glucopyranosylamine (329 mg, 1.82 mmol) dissolved in methanol/water (2 ml/1.5 ml) was added. As a result, a white solid was precipitated. The mixture was warmed to room temperature and stirred for 10 min. The solid obtained by filtration was slurry scrubbed with diethyl ether and methanol in this order to give Z-Arg(Z)₂-Glc (530 mg, 0.72 mmol, yield 60%) as a pale-yellow powder.

¹H-NMR (400 MHz, DMSO-d₆) δ: 1.47-1.61 (4H, m), 3.03-3.12 (m, 3H), 3.81-3.89 (m, 2H), 4.03-4.08 (m, 1H), 4.44 (t, 1H, J=5.7 Hz), 4.70 (t, 1H, J=8.9 Hz), 4.83 (d, 1H, J=5.5 Hz), 4.88 (d, 1H, J=5.0 Hz), 4.98-5.04 (m, 4H), 5.22 (s, 2H), 7.29-7.43 (m, 15H).

ESIMS (m/z): 760.1 ([M+Na]⁺), 736.1 ([M−H]⁻).

### (2) Arg-Glc; N-(L-arginyl)-β-D-glucopyranosylamine

In the same manner as in Example 8, step (2), Arg-Glc (149 mg, 0.46 mmol, yield 84%) was obtained as a white powder from Z-Arg(Z)₂-Glc (202 mg, 0.27 mmol).

¹H-NMR (400 MHz, D₂O) δ: 1.33-1.63 (m, 4H), 3.07-3.12 (m, 2H), 3.30-3.67 (m, 2H), 3.42-3.47 (m, 2H), 3.61-3.67 (m, 2H), 3.79 (dd, 1H, J=2.2 Hz), 4.90 (d, 1H, J=9.0 Hz).

ESIMS (m/z): 336.1 ([M+H]⁺), 358.1 ([M+Na]⁺), 333.9 ([M−H]⁻).

### Example 21

### DOPA-Glc; N-(3,4-dihydroxy-L-phenylalanyl)-β-D-glucopyranosylamine

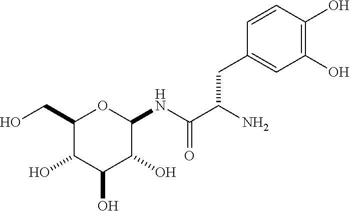 [SMILES: N[C@@H](Cc1ccc(O)c(O)c1)C(=O)N[C@@H]1O[C@H](CO)[C@@H](O)[C@H](O)[C@H]1O]

### (1) DOPA-OMe; methyl 3,4-dihydroxy-L-phenylalaninate hydrochloride

Methanol (50 ml) was cooled to −5° C. in a thermostatic bath, and thionyl chloride (5 ml, 68.9 mmol) was added dropwise. Then, 3,4-dihydroxy-L-phenylalanine (L-DOPA) (10.0 g, 50.7 mmol) was added by small portions, and the mixture was stirred for 5 min. The mixture was warmed to room temperature, heated to 50° C., and stirred for 14 hr. Then, the reaction solution was concentrated to give DOPA-OMe hydrochloride (14.3 g, 67.7 mmol, yield quant.) as an oil.

¹H-NMR (400 MHz, CD₃OD) δ: 3.04 (dd, 1H, J=7.4 Hz, 14.5 Hz), 3.13 (dd, 1H, J=5.8 Hz, 14.5 Hz), 3.84 (s, 3H), 4.22-4.25 (m, 1H), 6.58 (dd, 1H, J=2.2 Hz, 8.0 Hz), 6.69 (d, 1H, J=2.1 Hz), 6.77 (d, 1H, J=8.0 Hz).

ESIMS (m/z): 212.7 ([M+H]⁺), 423.2 ([2 M+H]⁺), 210.2 ([M−H]⁻), 241.1 ([M+Cl]⁻).

### (2) Z-DOPA-OMe; methyl N-(benzyloxycarbonyl)-3,4-dihydroxy-L-phenylalaninate

DOPA-OMe (1.26 g, 5.11 mmol) was dissolved in N,N-dimethylformamide (10 ml), triethylamine (1.57 ml, 11.2 mmol) was added and the mixture was cooled in an ice bath. To this solution was added benzyl chloroformate (0.802 ml, 5.62 mmol) and the mixture was warmed to room temperature and stirred for 1.5 hr. 1.5 N Hydrochloric acid (40 ml) was added and the mixture was extracted twice with diethyl ether (40 ml). The organic layer was washed with 15% brine (40 ml), and dried over magnesium sulfate. The desiccant was filtered off, and the filtrate was concentrated under reduced pressure, and the residue was purified by silica gel column chromatography (gradient; ethyl acetate:hexane=1:19→9:11) to give Z-DOPA-OMe (593 mg, 1.72 mmol, yield 34%) as a transparent oil.

¹H-NMR (400 MHz, CDCl₃) δ: 2.91-3.04 (m, 2H), 3.72 (s, 3H), 4.52-4.62 (m, 1H), 5.09 (d, 2H, J=6.6 Hz), 5.28 (d, 1H, J=8.1 Hz), 5.58 (s, 1H), 5.66 (s, 1H), 6.50 (dd, 1H, J=1.6 Hz, 8.0 Hz), 6.56 (br, 1H), 6.72 (d, 1H, J=8.1 Hz), 7.30-7.37 (m, 5H).

ESIMS (m/z): 344.1[M−H]⁻, 689.4 [2 M−H]⁻.

### (3) Z-DOPA(OBn)₂-OMe; methyl N-(benzyloxycarbonyl)-3,4-bis(benzyloxy)-L-phenylalaninate

Z-DOPA-OMe (593 mg, 1.72 mmol) was dissolved in N,N-dimethylformamide (10 ml), and the mixture was cooled in an ice bath. To this solution were added potassium carbonate (713 mg, 5.16 mmol), and benzyl bromide (0.470 ml, 3.96 mmol), and the mixture was warmed to room temperature, heated to 50° C. and stirred for 1 hr. Water (80 ml) was added, and the mixture was extracted twice with diethyl ether (50 ml). The organic layer was washed with 15% brine (40 ml), and dried over magnesium sulfate. The desiccant was filtered off, and the filtrate was concentrated under reduced pressure to give Z-DOPA(OBn)₂-OMe (800 mg, 1.52 mmol, yield 88%) as a white powder.

¹H-NMR (400 MHz, CDCl₃) δ: 2.96-3.05 (m, 2H), 3.64 (s, 3H), 4.59-4.62 (m, 1H), 5.07-5.12 (m, 6H), 6.60 (dd, 1H, J=2.0 Hz, 8.1 Hz), 6.70 (d, 1H, J=1.7 Hz), 6.83 (d, 1H, J=8.2 Hz), 7.28-7.43 (m, 15H).

ESIMS (m/z): 526.3 ([M+H]⁺), 543.3 ([M+NH₄]⁺), 548.2 ([M+Na]⁺), 564.2 ([M+K]⁺).

### (4) Z-DOPA(OBn)₂; N-(benzyloxycarbonyl)-3,4-bis(benzyloxy)-L-phenylalanine

Z-DOPA(OBn)₂-OMe (416 mg, 0.793 mmol) was dissolved in methanol/tetrahydrofuran (1 ml/2 ml), and the mixture was cooled in an ice bath. To this solution were added 1N aqueous lithium hydroxide solution (1.5 ml) and water (9 ml), and the mixture was warmed to room temperature and stirred for 1 hr. The mixture was neutralized with Amberlite-H resin and the resin was filtered off. The residue was concentrated to give Z-DOPA(OBn)₂ (405 mg, 0.793 mmol, yield quant.) as a white powder.

¹H-NMR (400 MHz, CDCl₃) δ: 2.96-3.09 (m, 2H), 4.57-4.64 (m, 1H), 5.01-5.14 (m, 6H), 6.64 (dd, 1H, J=2.1 Hz, 8.2 Hz), 6.70 (br, 1H), 6.83 (d, 1H, J=8.2 Hz), 7.28-7.43 (m, 15H).

ESIMS (m/z): 512.2 ([M+H]⁺), 529.2 ([M+NH₄]⁺), 510.1 ([M−H]⁻).

### (5) Z-DOPA(OBn)₂-Glc; N—(N-(benzyloxycarbonyl)-3,4-bis(benzyloxy)-L-phenylalanyl)-β-D-glucopyranosylamine

Z-DOPA(OBn)₂ (405 mg, 0.793 mmol) was dissolved in tetrahydrofuran (5 ml) at room temperature, and the mixture was cooled in an ice bath. To this solution were added triethylamine (0.221 ml, 1.59 mmol) and pivaloyl chloride (0.125 ml, 1.03 mmol) and the mixture was stirred for 30 min. Then, D-glucopyranosylamine (185 mg, 1.03 mmol) dissolved in methanol/water (2 ml/0.5 ml) was added. The mixture was warmed to room temperature and stirred for 2 hr. The reaction solution was concentrated under reduced pressure, and the residue was slurry scrubbed with water and diethyl ether in this order to give Z-DOPA(OBn)₂-Glc (371 mg, 0.55 mmol, yield 70%) as a white powder.

¹H-NMR (400 MHz, CDCl₃) δ: 2.76-2.82 (m, 1H), 3.11 (dd, 1H, J=4.6 Hz, 14.2 Hz), 3.30-3.45 (m, 3H), 3.68 (dd, 1H, J=3.4 Hz, 11.5 Hz), 3.82-3.85 (m, 1H), 4.39-4.42 (m, 1H), 5.08 (d, 1H, J=8.9 Hz), 6.80-6.84 (m, 1H), 6.94 (d, 1H, J=8.2 Hz), 7.02 (d, 1H, J=1.8 Hz), 7.25-7.48 (m, 15H).

ESIMS (m/z): 671.0 ([M−H]⁻).

### (6) DOPA-Glc; N-(3,4-dihydroxy-L-phenylalanyl)-β-D-glucopyranosylamine

In the same manner as in Example 2, step (2), deprotection of Z-DOPA(OBn)₂-Glc (371 mg, 0.55 mmol) was performed. Purification by ODS column chromatography gave DOPA-Glc (56.7 mg, 0.158 mmol, yield 30%) as a brown powder.

¹H-NMR (400 MHz, CDCl₃) δ: 2.77-2.92 (m, 2H), 3.27-3.49 (m, 4H), 3.59-3.65 (m, 1H), 3.71-3.80 (m, 2H), 4.86 (d, 1H, J=9.2 Hz), 6.61 (dd, 1H, J=2.0 Hz, 8.1 Hz), 6.68 (d, 1H, J=1.9 Hz), 6.76 (d, 1H, J=8.1 Hz).

ESIMS (m/z): 359.1 ([M+H]⁺), 381.1 ([M+Na]⁺), 717.3 ([2 M+H]⁺), 739.3. ([2 M+Na]⁺), 357.1 ([M−H]⁻), 715.3 ([2 M−H]⁻).

### Experimental Example 1

### Sensory Evaluation

Since leucine has a unique bitter taste, Glc-Leu and Glc-Leu-Glc were examined by sensory evaluation for masking effect on their bitter taste. Three test subjects A, B, C took 0.1 ml of a solution of food additive leucine dissolved in water at a concentration of 0.5% (5000 ppm) with a micropipette, dropped the solution on the tongue, and spit it out to confirm the level of the bitter taste of leucine. Sequentially, the three test subjects A, B, C took 0.1 ml of a solution of Glc-Leu or Glc-Leu-Glc dissolved in water at a concentration of 0.5% (5000 ppm) with a micropipette, dropped the solution on the tongue, and spit it out to compare the level of the bitter taste with that of leucine confirmed earlier. The results are as follows and none of the test subjects felt the bitter taste confirmed with leucine.

TABLE 1
sensory evaluation of glycoamino acid

| glycoleucine | test subject A | test subject B | test subject C |
|---|---|---|---|
| Glc-Leu | No bitter taste | No bitter taste | No bitter taste |
| | | faintly sweet | |
| Glc-Leu-Glc | No bitter taste | No bitter taste | No bitter taste |
| | | faintly sweet | |

### Experimental Example 2

### Enzyme Evaluation

Leu-Glc (10 mg) was dissolved in water (1 ml), pronase (0.1% aqueous solution, 100 μl) was added, and the mixture was stirred in a hot-water bath at 37° C. The mixture was diluted 10-fold with 1% aqueous phosphoric acid solution, and analyzed by HPLC. The results are shown in FIG. 1. From 2 min after the enzyme addition, about 50% of leucine was liberated, and Leu-Glc almost disappeared 30 min later.

HPLC analysis conditions were as described below.

column: CAPCELLPAK MG (4.6×250 mm, 5 μm)
column temperature: 40° C.
mobile phase: A: 100 mM KH₂PO₄, 5 mM sodium 1-octanesulfonate (pH 2.2)
B: acetonitrile
eluent: A/B=9/1 isocratic
flow rate: 1.5 ml/min
detection: photodiode array detector measurement wavelength 210 nm
injection volume: 10 μL

### Experimental Example 3

### Artificial Bowel Fluid Evaluation

Pancreatin was dissolved in 2nd fluid described in the dissolution test of the Japanese Pharmacopoeia, 15th Edition, (1 volume of pH 6.8 phosphate buffer added with 1 volume of water) at a concentration of 4% to give an artificial bowel fluid.

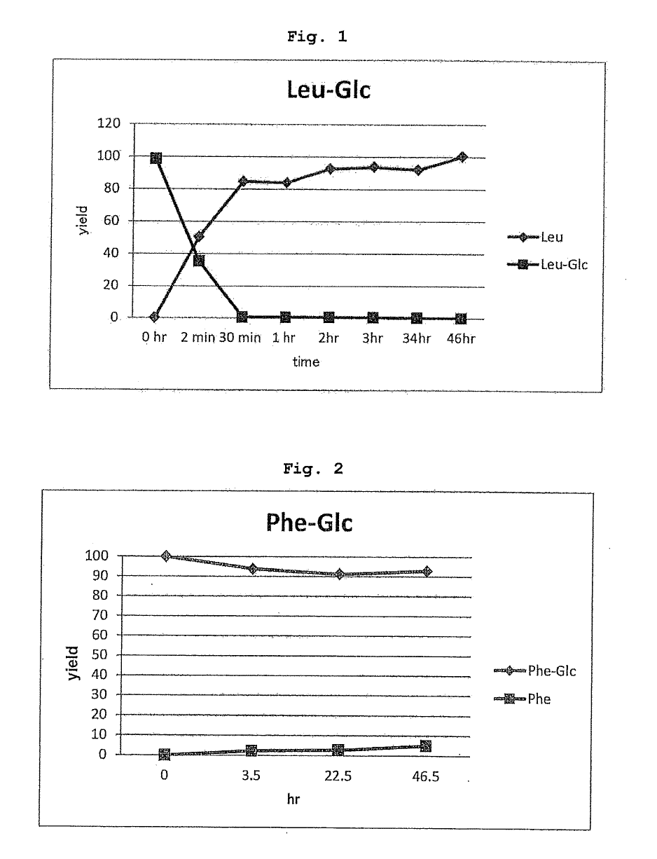

Glc-Phe (1.0 mg) was dissolved in the artificial bowel fluid (1 ml), stirred in a hot-water bath at 37° C., and analyzed by HPLC. The results thereof are shown in FIG. 2. 2% of Phe was liberated 3.5 hr later, 3% of Phe was liberated 22 hr later and 5% of Phe was liberated 46.5 hr later.

HPLC conditions were as described below.

column: CAPCELLPAK MG (4.6×250 mm, 5 μm)
column temperature: 40° C.
mobile phase: A: 100 mM KH₂PO₄, 5 mM sodium 1-octanesulfonate (pH 2.2)
B: acetonitrile
eluent: A/B=9/1 isocratic
flow rate: 1.5 ml/min
detection: photodiode array detector measurement wavelength 210 nm
injection volume: 10 μL

### Experimental Example 4

### Dissolution Rate Evaluation

Val, Ile, Leu or glycoamino acid corresponding thereto (Val-Glc, Ile-Glc, Leu-Glc) were each added to stirring water (25 ml, inside temperature 32° C.) in a hot-water bath at 35° C., and the dissolution rate was measured. The amount of the sample added and the measurement results are as shown in Tables 2 and 3 (n=1). As compared to Val, Ile and Leu, Val-Glc, Ile-Glc and Leu-Glc were dissolved 4-19 times faster in equal weight and 2-19 times faster in equimolar amount.

TABLE 2
dissolution rate of equimolar quantity of amino
acid and glycoamino acid corresponding thereto

| | | | | glycoamino | |
|---|---|---|---|---|---|
| | | amino acid (XXX) | | acid (XXX-Glc) | |
| | added molar | added | disso- | added | disso- |
| | quantity/ | weight/25 | lution | weight/25 | lution |
| XXX | 25 ml water | ml water | rate | ml water | rate |
| Val | 1.80 mmol | 211 mg | 1 min | 500 mg | 33 sec |
| | | | 20 sec | | |
| | | | (80 sec) | | |
| Ile | 1.71 mmol | 224 mg | 4 min | 500 mg | 15 sec |
| | | | 40 sec | | |
| | | | (280 sec) | | |
| Leu | 1.03 mmol | 135 mg | 3 min | 300 mg | 21 sec |
| | | | 11 sec | | |
| | | | (191 sec) | | |

TABLE 3
dissolution rate of equal weight of amino acid
and glycoamino acid corresponding thereto

| | | | | glycoamino | |
|---|---|---|---|---|---|
| | | amino acid (XXX) | | acid (XXX-Glc) | |
| | added | added molar | disso- | added molar | disso- |
| | weight/25 | quantity/ | lution | quantity/ | lution |
| XXX | ml water | 25 ml water | rate | 25 ml water | rate |
| Val | 500 mg | 4.27 mmol | 2 min | 1.79 mmol | 33 sec |
| | | | 19 sec | | |
| | | | (139 sec) | | |
| Ile | 500 mg | 3.81 mmol | 4 min | 1.71 mmol | 15 sec |
| | | | 45 sec | | |
| | | | (285 sec) | | |
| Leu | 300 mg | 2.29 mmol | 5 min | 1.03 mmol | 21 sec |
| | | | 30 sec | | |
| | | | (330 sec) | | |

### Experimental Example 5

### Solubility Evaluation

Val, Ile, Leu, Tyr and glycoamino acid corresponding thereto (Val-Glc, Ile-Glc, Leu-Glc, Tyr-Glc) were each added to water (1 ml) in a thermostatic bath at 25° C. until they remained undissolved, the mixture was stirred for 2 days and the solubility was measured. The concentration was measured by HPLC. As a result, the solubility of each of Val-Glc, Ile-Glc and Leu-Glc increased 2- to 12-fold as compared to that of Val, Ile and Leu. The solubility of Tyr-Glc was markedly improved by 178 times as compared to Tyr. Similarly, the solubility of DOPA and DOPA-Glc was measured. DOPA-Glc showed extremely high solubility, and was dissolved even at weight concentration 93.8 g/100 g water. Therefrom it was suggested that DOPA-Glc has a solubility not less than 135 times that of DOPA. Furthermore, the solubility of DOPA and DOPA-Glc was similarly measured using water (0.5 ml) in a thermostatic tank at 25° C. When about 1.5 g of DOPA-Glc was added, they were dissolved in water; however, the viscosity thereof was high at this time point and stirring was difficult. Therefore, the samples were diluted, and solubility was measured by HPLC. As a result, the solubility of DOPA-Glc was not less than 690-fold as compared to that of DOPA.

TABLE 4

| | | amino acid-converted |
|---|---|---|
| | weight concentration | weight concentration* |
| | (g/100 g water) | (g/100 g water) |
| Val-Glc | 33.5 | 14.1 |
| Val | 5.8 | 5.8 |
| Ile-Glc | 32.8 | 14.7 |
| Ile | 4.1 | 4.1 |
| Leu-Glc | 63 | 28.3 |
| Leu | 2.4 | 2.4 |
| Tyr-Glc | 16.8 | 8.92 |
| Tyr | 0.05 | 0.05 |
| DOPA-Glc | >392 | >215 |
| DOPA | 0.31 | 0.31 |

*The amino acid-converted weight concentration of glycoamino acid is the weight concentration of amino acid corresponding to the number of moles of dissolved glycoamino acid, and the amino acid-converted weight concentration of amino acid is equal to the weight concentration of amino acid.

### Experimental Example 6

### Animal Evaluation Results

Leu, Val, Ile and glycoamino acid corresponding thereto (Leu-Glc, Val-Glc, Ile-Glc) were each dissolved or suspended in distilled water to a given dose and orally administered to male 13-week-old SD rats (Japan Charles River) that was fasted overnight. Blood samples were collected from the rat tail vein before administration and 15 min, 30 min, 60 min, 90 min, 120 min after administration and partly 180 min and 300 min after administration. After separation into plasma, protein elimination and ultrafiltration with 15% sulfosalicylic acid solution was performed. The filtrate was mixed with 0.02 mmol/L hydrochloric acid at 1:1, analyzed by an amino acid analyzer (JEOL Ltd.), and blood amino acid concentration was determined.

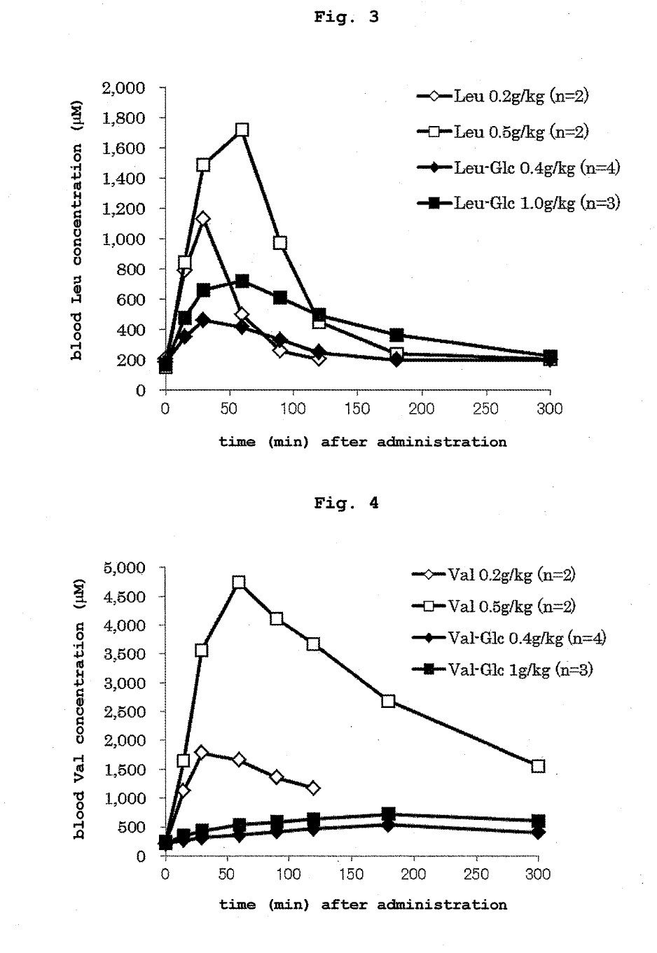

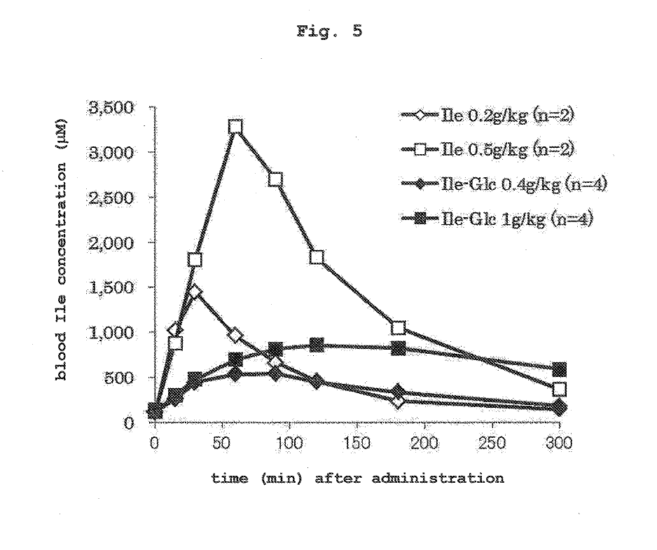

FIG. 3 shows changes in blood Leu concentration by Leu or Leu-Glc administration, FIG. 4 shows changes in blood Val concentration by Val or Val-Glc administration, and FIG. 5 shows changes in blood Ile concentration after Ile or Ile-Glc administration. The blood Leu, Val and Ile concentrations increased by oral administration of Leu-Glc, Val-Glc and Ile-Glc. Therefrom it was shown that the oral administration of Leu-Glc, Val-Glc and Ile-Glc increases the blood concentration of each amino acid as the mother nucleus.

### Example 22

According to the disclosure of JP-A-8-73351, the amino acid composition (16.42 parts) shown in the following Table 5, safflower oil (1.43 parts), purification Japanese basil oil (0.57 part), dextrin (76.45 parts) and vitamins and minerals (5.13 parts) are mixed to prepare a nutrition composition for inflammatory bowel diseases.

TABLE 5

| amino acid | composition (g/total amount |
|---|---|
| or amino | 100 g of amino acid or amino |
| acid precursor | acid precursor in Table) |
| Ile-Glc | 5.96 |
| Leu-Glc | 11.93 |
| Val-Glc | 5.96 |
| Lys-Glc | 5.48 |
| Met-Glc | 3.48 |
| Phe-Glc | 6.46 |
| Thr-Glc | 3.98 |
| Trp-Glc | 1.49 |
| Ala-Glc | 5.13 |
| Arg-Glc | 9.95 |
| Asp-Glc | 5.54 |
| Gln-Glc | 24.85 |
| Gly-Glc | 2.00 |
| His-Glc | 1.99 |
| Pro-Glc | 2.98 |
| Ser-Glc | 1.99 |
| Tyr-Glc | 0.83 |

### INDUSTRIAL APPLICABILITY

A glycoamino acid wherein a group represented by the formula G²-NH— wherein G² is as defined above is introduced into a carboxy group of amino acid, or a salt thereof, shows improvement in the properties (particularly water-solubility, stability in water, bitter taste etc.) that the amino acid itself has, and the glycoamino acid or a salt thereof can be an amino acid precursor which is converted to amino acid in vivo, since the above-mentioned group represented by the formula G²-NH— is detached from amino acid in vivo etc. Therefore, the compound for an amino acid precursor of the present invention is suitable for ingestion, and also suitable as an aqueous composition or for oral application. Using such compound for an amino acid precursor of the present invention having improved water-solubility even in amino acid having comparatively high water-solubility, the broad utility of amino acid in the preparation of an aqueous composition or liquid composition for oral ingestion, and the like is markedly improved.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A compound represented by formula (I): 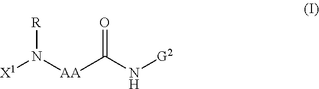
wherein
N(R)(X¹)-AA-C(═O) is an amino acid residue;
X¹ is a hydrogen atom, or a group represented by G¹-O—C(O)—, wherein G¹ is a sugar residue wherein none of the hydroxyl groups are protected or modified;
G² is a sugar residue wherein none of the hydroxyl groups are protected or modified; and
R is a hydrogen atom or an alkyl group,
or a salt thereof.

2. The compound or salt according to claim 1, wherein the sugar for said sugar residue for G¹ or G² is a monosaccharide.

3. The compound or salt according to claim 1, wherein the sugar for the sugar residue for G² is glucose.

4. The compound or salt according to claim 1, wherein the sugar for the sugar residue for G¹ is glucose, glucosamine, or N-acetylglucosamine.

5. The compound or salt according to claim 1, wherein R is a hydrogen atom.

6. The compound or salt according to claim 1, wherein X¹ is a hydrogen atom and R is a hydrogen atom.

7. The compound or salt according to claim 6, wherein the sugar for the sugar residue for G² is glucose.

8. The compound or salt according to claim 1, wherein the amino acid of said amino acid residue is an α-amino acid.

9. The compound or salt according to claim 1, wherein the amino acid of said amino acid residue is valine, leucine, isoleucine, phenylalanine, tyrosine or 3,4-dihydroxyphenylalanine.

10. The compound or salt according to claim 1, which is converted to amino acid in vivo.

11. The compound salt precursor according to claim 1, which is suitable for ingestion.

12. A composition, comprising a compound or salt according claim 1 and a carrier, wherein said composition is suitable for ingestion.

13. The composition according to claim 12, which is suitable for oral application.

14. A method of suppressing a bitter taste of an amino acid, comprising introducing a group represented by formula G²-NH—, wherein G² is a sugar residue wherein none of the hydroxyl groups are protected or modified, into a carboxy group of said amino acid.

15. The method according to claim 14, wherein the sugar for said sugar residue for G² is a monosaccharide.

16. The method according to claim 14, wherein the sugar for said sugar residue for G² is glucose.

17. The method according to claim 14, wherein said amino acid is an α-amino acid.

18. The method according to claim 14, wherein said amino acid is valine, leucine, or isoleucine.

19. The method according to claim 14, wherein the amino acid, wherein a group represented by the formula G²-NH— is introduced into a carboxy group, is converted to an amino acid in vivo.

20. A compound represented by: 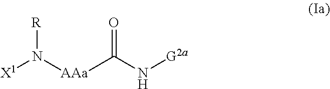
wherein
N(R)(X¹)-AAa-C(═O) is a residual group of an amino acid selected from the group consisting of valine, leucine, isoleucine, tyrosine, and 3,4-dihydroxyphenylalanine;
X¹ is a hydrogen atom, or a group represented by G¹-O—C(O)—, wherein G¹ is a sugar residue wherein none of the hydroxyl groups are protected or modified;
G^{2a} is a monosaccharide residue wherein none of the hydroxyl groups are protected or modified; and
R is a hydrogen atom or an alkyl group
or a salt thereof.

21. The compound or salt according to claim 20, wherein the sugar for said monosaccharide residue for G^{2a} is glucose.

22. The compound or salt according to claim 20, wherein the sugar for said sugar residue for G¹ is a monosaccharide.

23. The compound or salt according to claim 20, wherein the sugar for said sugar residue for G¹ is glucose, glucosamine, or N-acetylglucosamine.

24. The compound or salt according to claim 20, wherein R is a hydrogen atom.

25. The compound or salt according to claim 20, wherein X¹ is a hydrogen atom and R is a hydrogen atom.

26. The compound or salt according to claim 25, wherein the sugar for said monosaccharide residue for G^{2a} is glucose.

27. The compound or salt according to claim 20, which is converted to amino acid in vivo.

28. A method of administering an amino acid, comprising administering a compound or salt according to claim 10 to a subject in need thereof.
